(19) United States
(12) Patent Application Publication
(10) Pub. No.: US 2024/0041401 A1
(43) Pub. Date: Feb. 8, 2024

(54) EAR-WEARABLE SYSTEM AND METHOD FOR DETECTING DEHYDRATION

(71) Applicant: Starkey Laboratories, Inc., Eden Prairie, MN (US)

(72) Inventors: Paul N. Reinhart, Minneapolis, MN (US); Andy S. Lin, Chanhassen, MN (US)

(21) Appl. No.: 18/268,836

(22) PCT Filed: Dec. 22, 2021
(86) PCT No.: PCT/US2021/064886

§ 371 Date: Jun. 21, 2023

Related U.S. Application Data
(60) Provisional application No. 63/130,194, filed on Dec. 23, 2020.

(51) Int. Cl. A61B 5/00; A61B 5/0205; A61B 5/11

(52) U.S. Cl. CPC A61B 5/4875; A61B 5/0205; A61B 5/4023; A61B 5/1101; A61B 5/4058; A61B 5/4205; A61B 5/746; A61B 5/7282; A61B 5/7267; A61B 5/6815; A61B 5/021

(57) ABSTRACT

Embodiments herein relate to ear-wearable systems and devices for detecting dehydration and related methods. In an embodiment, an ear-wearable dehydration monitoring system is included having a control circuit, a microphone, a power supply, and a sensor package, wherein the sensor package is in electrical communication with the control circuit. The ear-wearable dehydration monitoring system is configured to process signals of one or more sensors of the sensor package to detect clinical symptoms of dehydration. Other embodiments are also included herein.

This application is being filed as a PCT International Patent application on Dec. 22, 2021 in the name of Starkey Laboratories, Inc., a U.S. national corporation, applicant for the designation of all countries, and Paul N. Reinhart, a U.S. Citizen, and Andy S. Lin, a U.S. Citizen, inventors for the designation of all countries, and claims priority to U.S. Provisional Patent Application No. 63/130,194, filed Dec. 23, 2020, the contents of which are herein incorporated by reference in its entirety.

### FIELD

Embodiments herein relate to ear-wearable systems and devices for detecting hydration levels, dehydration and related methods.

### BACKGROUND

Dehydration represents a significant health issue among elderly people. While anyone can experience dehydration, older individuals are physiologically more susceptible due to changes in bodily fluid reserves, renal function, and thirst response. Older individuals also frequently have medical conditions and/or take medications that further dehydrate the body. As a result, the prevalence of dehydration among elderly individuals is approximately 20-30% and approximately 50% of elderly who are admitted to a hospital are in a state of dehydration. In severe cases of dehydration, mortality may be greater than 50% due to dehydration and/or related complications.

### SUMMARY

Embodiments herein relate to ear-wearable systems and devices for detecting hydration levels, dehydration and related methods. In a first aspect, an ear-wearable hydration level monitoring system is included having a control circuit, a microphone, wherein the microphone is in electrical communication with the control circuit, a power supply, wherein the power supply is in electrical communication with the control circuit, and a sensor package, wherein the sensor package is in electrical communication with the control circuit and wherein the ear-wearable hydration level monitoring system is configured to process signals of one or more sensors of the sensor package to detect clinical symptoms of hydration levels.

In a second aspect, in addition to one or more of the preceding or following aspects, or in the alternative to some aspects, the sensor package can include a motion sensor.

In a third aspect, in addition to one or more of the preceding or following aspects, or in the alternative to some aspects, the sensor package can include at least one selected from the group consisting of a photoplethysmography sensor, a temperature sensor, and a motion sensor.

In a fourth aspect, in addition to one or more of the preceding or following aspects, or in the alternative to some aspects, the sensor package can include at least one selected from the group consisting of a photoplethysmography (PPG) sensor, a electrocardiography (ECG) sensor, a temperature sensor, an electromyography (EMG) sensor, a motion sensor, an electroencephalography (EEG) sensor, and a glucose sensor.

In a fifth aspect, in addition to one or more of the preceding or following aspects, or in the alternative to some aspects, the clinical symptoms of hydration level including one or more of rapid shallow breathing, increased pulse, low blood pressure, dizziness, change in voice quality, increased temperature.

In a sixth aspect, in addition to one or more of the preceding or following aspects, or in the alternative to some aspects, the changes in voice quality include changes in tonal properties.

In a seventh aspect, in addition to one or more of the preceding or following aspects, or in the alternative to some aspects, the ear-wearable hydration level monitoring system is configured to receive data from at least one external sensor.

In an eighth aspect, in addition to one or more of the preceding or following aspects, or in the alternative to some aspects, the external sensor is selected from the group consisting of a humidity sensor, an ambient temperature sensor, a weight sensor, and a sensor disposed on a charging device for the ear-wearable hydration level monitoring system.

In a ninth aspect, in addition to one or more of the preceding or following aspects, or in the alternative to some aspects, the ear-wearable hydration level monitoring system is configured to process signals of the microphone to detect signs of hydration level.

In a tenth aspect, in addition to one or more of the preceding or following aspects, or in the alternative to some aspects, the signs of hydration level include smacking or licking lips.

In an eleventh aspect, in addition to one or more of the preceding or following aspects, or in the alternative to some aspects, the signs of hydration level include changes in voice pitch and/or tremor.

In a twelfth aspect, in addition to one or more of the preceding or following aspects, or in the alternative to some aspects, the signs of hydration level include rapid shallow breathing.

In a thirteenth aspect, in addition to one or more of the preceding or following aspects, or in the alternative to some aspects, the signs of hydration level include dysphonia.

In a fourteenth aspect, in addition to one or more of the preceding or following aspects, or in the alternative to some aspects, the ear-wearable hydration level monitoring system is configured to issue an alert when hydration level clinical symptoms cross a threshold value.

In a fifteenth aspect, in addition to one or more of the preceding or following aspects, or in the alternative to some aspects, the threshold value is dynamically set based on factors including one or more of an ambient temperature, an ambient humidity, and activity levels of the device wearer.

In a sixteenth aspect, in addition to one or more of the preceding or following aspects, or in the alternative to some aspects, the ear-wearable hydration level monitoring system is configured to identify drinking events based at least in part on signals from the microphone and record the same.

In a seventeenth aspect, in addition to one or more of the preceding or following aspects, or in the alternative to some aspects, the ear-wearable hydration level monitoring system is configured to issue an alert if a number of identified drinking events over a defined time period change by at least a threshold value.

In an eighteenth aspect, in addition to one or more of the preceding or following aspects, or in the alternative to some aspects, the ear-wearable hydration level monitoring system is configured to issue an alert when hydration level clinical symptoms cross a threshold value for at least a threshold period of time.

In a nineteenth aspect, in addition to one or more of the preceding or following aspects, or in the alternative to some aspects, further can include a first unit, wherein the first unit is configured to be wearable about a first ear, and a second unit, wherein the second unit is configured to be wearable about a second ear, wherein signals are exchanged between the first unit and the second unit.

In a twentieth aspect, in addition to one or more of the preceding or following aspects, or in the alternative to some aspects, the ear-wearable hydration level monitoring system can be configured to classify an observed pattern representing signals from the microphone and the sensor package into a scale of hydration levels using a machine learning derived algorithm.

In a twenty-first aspect, an ear-wearable hydration level monitoring system is included having a control circuit, a microphone, wherein the microphone is in electrical communication with the control circuit, a power supply, wherein the power supply is in electrical communication with the control circuit, a sealing member attached to a structure, and a humidity sensor attached to the structure, wherein the humidity sensor is configured to measure humidity within an ear canal of a wearer of the ear-wearable hydration level monitoring system between the sealing member and a tympanic membrane of the wearer.

In a twenty-second aspect, in addition to one or more of the preceding or following aspects, or in the alternative to some aspects, the ear-wearable hydration level monitoring system is configured to receive data from at least one external sensor.

In a twenty-third aspect, in addition to one or more of the preceding or following aspects, or in the alternative to some aspects, the external sensor is selected from the group consisting of a humidity sensor, an ambient temperature sensor, a weight sensor, and a sensor disposed on a charging device for the ear-wearable hydration level monitoring system.

In a twenty-fourth aspect, in addition to one or more of the preceding or following aspects, or in the alternative to some aspects, the sealing member can include a sealing dome.

In a twenty-fifth aspect, in addition to one or more of the preceding or following aspects, or in the alternative to some aspects, the sealing member can include a sealing baffle.

In a twenty-sixth aspect, in addition to one or more of the preceding or following aspects, or in the alternative to some aspects, wherein the sealing baffle is mounted on a receiver.

In a twenty-seventh aspect, in addition to one or more of the preceding or following aspects, or in the alternative to some aspects, further can include a sensor package, wherein the sensor package is in electrical communication with the control circuit.

In a twenty-eighth aspect, in addition to one or more of the preceding or following aspects, or in the alternative to some aspects, the sensor package can include a motion sensor.

In a twenty-ninth aspect, in addition to one or more of the preceding or following aspects, or in the alternative to some aspects, the sensor package can include at least one selected from the group consisting of a photoplethysmography sensor, a temperature sensor, and a motion sensor.

In a thirtieth aspect, in addition to one or more of the preceding or following aspects, or in the alternative to some aspects, the sensor package can include at least one selected from the group consisting of a photoplethysmography (PPG) sensor, a electrocardiography (ECG) sensor, a temperature sensor, an electromyography (EMG) sensor, a motion sensor, an electroencephalography (EEG) sensor, and a glucose sensor.

In a thirty-first aspect, in addition to one or more of the preceding or following aspects, or in the alternative to some aspects, the ear-wearable hydration level monitoring system is configured to issue an alert when ear canal humidity crosses a threshold value.

In a thirty-second aspect, in addition to one or more of the preceding or following aspects, or in the alternative to some aspects, the threshold value is dynamically set based on factors including one or more of an ambient temperature, an ambient humidity, and activity levels of the device wearer.

In a thirty-third aspect, in addition to one or more of the preceding or following aspects, or in the alternative to some aspects, the ear-wearable hydration level monitoring system is configured to identify drinking events based at least in part on signals from the microphone and record the same.

In a thirty-fourth aspect, in addition to one or more of the preceding or following aspects, or in the alternative to some aspects, the ear-wearable hydration level monitoring system is configured to issue an alert if a number of identified drinking events over a defined time period change by at least a threshold value.

In a thirty-fifth aspect, a method of monitoring an individual for hydration level using an ear-wearable monitoring system is included, the method including gathering signals with a microphone, gathering signals with a sensor package, and processing signals of the microphone and the sensor package to detect clinical symptoms of hydration level.

In a thirty-sixth aspect, in addition to one or more of the preceding or following aspects, or in the alternative to some aspects, the clinical symptoms of hydration level including one or more of rapid shallow breathing, increased pulse, low blood pressure, dizziness, change in voice quality, increased temperature.

In a thirty-seventh aspect, in addition to one or more of the preceding or following aspects, or in the alternative to some aspects, the changes in voice quality include changes in tonal properties.

In a thirty-eighth aspect, in addition to one or more of the preceding or following aspects, or in the alternative to some aspects, the method can further include processing signals of the microphone to detect signs of hydration level.

In a thirty-ninth aspect, in addition to one or more of the preceding or following aspects, or in the alternative to some aspects, the signs of hydration level include dysphonia.

In a fortieth aspect, in addition to one or more of the preceding or following aspects, or in the alternative to some aspects, the signs of hydration level include rapid shallow breathing.

In a forty-first aspect, in addition to one or more of the preceding or following aspects, or in the alternative to some aspects, the signs of hydration level include changes in voice pitch and/or tremor.

In a forty-second aspect, in addition to one or more of the preceding or following aspects, or in the alternative to some aspects, the signs of hydration level include smacking or licking lips.

In a forty-third aspect, in addition to one or more of the preceding or following aspects, or in the alternative to some aspects, the method can further include issuing an alert when hydration level clinical symptoms cross a threshold value.

In a forty-fourth aspect, in addition to one or more of the preceding or following aspects, or in the alternative to some aspects, the threshold value is dynamically set based on factors including one or more of an ambient temperature, an ambient humidity, and activity levels of the device wearer.

In a forty-fifth aspect, in addition to one or more of the preceding or following aspects, or in the alternative to some aspects, the method can further include identifying drinking events based at least in part on signals from the microphone and record the same.

In a forty-sixth aspect, in addition to one or more of the preceding or following aspects, or in the alternative to some aspects, wherein identifying drinking events based at least in part on signals from the microphone and record the same further includes issuing an alert if a number of identified drinking events over a defined time period change by at least a threshold value.

In a forty-seventh aspect, in addition to one or more of the preceding or following aspects, or in the alternative to some aspects, the method can further include issuing an alert when hydration level clinical symptoms cross a threshold value for at least a threshold period of time.

In a forty-eighth aspect, in addition to one or more of the preceding or following aspects, or in the alternative to some aspects, the method can further include classifying an observed pattern representing signals from the microphone and the sensor package into a scale of hydration level severities using a machine learning derived algorithm.

In a forty-ninth aspect, a method of monitoring ear canal humidity to detect hydration level of an individual is included, the method including sealing off a portion of an individual's ear canal, measuring humidity within the sealed off portion of the ear canal, and evaluating the measured humidity to detect hydration level.

In a fiftieth aspect, in addition to one or more of the preceding or following aspects, or in the alternative to some aspects, the method can further include issuing an alert when humidity values cross a threshold value.

In a fifty-first aspect, in addition to one or more of the preceding or following aspects, or in the alternative to some aspects, the method can further include issuing an alert when humidity values cross a threshold value for at least a threshold period of time.

In a fifty-second aspect, in addition to one or more of the preceding or following aspects, or in the alternative to some aspects, the threshold value is dynamically set based on factors including one or more of an ambient temperature, an ambient humidity, and activity levels of the device wearer.

In a fifty-third aspect, in addition to one or more of the preceding or following aspects, or in the alternative to some aspects, the method can further include identifying drinking events based at least in part on signals from a microphone and record the same.

In a fifty-fourth aspect, in addition to one or more of the preceding or following aspects, or in the alternative to some aspects, the method can further include issuing an alert if a number of identified drinking events over a defined time period change by at least a threshold value.

In a fifty-fifth aspect, in addition to one or more of the preceding or following aspects, or in the alternative to some aspects, the method can further include gathering signals with a microphone.

In a fifty-sixth aspect, in addition to one or more of the preceding or following aspects, or in the alternative to some aspects, the method can further include gathering signals with a sensor package.

In a fifty-seventh aspect, in addition to one or more of the preceding or following aspects, or in the alternative to some aspects, the method can further include processing signals of the microphone and the sensor package to detect clinical symptoms of hydration level.

In a fifty-eighth aspect, in addition to one or more of the preceding or following aspects, or in the alternative to some aspects, the clinical symptoms of hydration level including one or more of rapid shallow breathing, increased pulse, low blood pressure, dizziness, change in voice quality, increased temperature.

In a fifty-ninth aspect, in addition to one or more of the preceding or following aspects, or in the alternative to some aspects, the changes in voice quality include changes in tonal properties.

In a sixtieth aspect, in addition to one or more of the preceding or following aspects, or in the alternative to some aspects, the method can further include processing signals of the microphone to detect signs of hydration level.

In a sixty-first aspect, in addition to one or more of the preceding or following aspects, or in the alternative to some aspects, the signs of hydration level include smacking or licking lips.

In a sixty-second aspect, in addition to one or more of the preceding or following aspects, or in the alternative to some aspects, the signs of hydration level include changes in voice pitch and/or tremor.

In a sixty-third aspect, in addition to one or more of the preceding or following aspects, or in the alternative to some aspects, the signs of hydration level include rapid shallow breathing.

In a sixty-fourth aspect, in addition to one or more of the preceding or following aspects, or in the alternative to some aspects, the signs of hydration level include dysphonia.

In a sixty-fifth aspect, an ear-wearable dehydration monitoring system can be included having a control circuit, a microphone, wherein the microphone can be in electrical communication with the control circuit, a power supply, wherein the power supply can be in electrical communication with the control circuit, and a sensor package, wherein the sensor package can be in electrical communication with the control circuit, wherein the ear-wearable dehydration monitoring system can be configured to process signals of one or more sensors of the sensor package to detect clinical symptoms of dehydration.

In a sixty-sixth aspect, in addition to one or more of the preceding or following aspects, or in the alternative to some aspects, the sensor package can include a motion sensor.

In a sixty-seventh aspect, in addition to one or more of the preceding or following aspects, or in the alternative to some aspects, the sensor package can include at least one selected from the group consisting of a photoplethysmography sensor, a temperature sensor, and a motion sensor.

In a sixty-eighth aspect, in addition to one or more of the preceding or following aspects, or in the alternative to some aspects, the sensor package can include at least one selected from the group consisting of a photoplethysmography (PPG) sensor, a electrocardiography (ECG) sensor, a temperature sensor, an electromyography (EMG) sensor, a motion sensor, an electroencephalography (EEG) sensor, and a glucose sensor.

In a sixty-ninth aspect, in addition to one or more of the preceding or following aspects, or in the alternative to some aspects, the clinical symptoms of dehydration can include one or more of rapid shallow breathing, increased pulse, low blood pressure, dizziness, change in voice quality, increased temperature.

In a seventieth aspect, in addition to one or more of the preceding or following aspects, or in the alternative to some aspects, the changes in voice quality include changes in tonal properties.

In a seventy-first aspect, in addition to one or more of the preceding or following aspects, or in the alternative to some aspects, the ear-wearable dehydration monitoring system can be configured to receive data from at least one external sensor.

In a seventy-second aspect, in addition to one or more of the preceding or following aspects, or in the alternative to some aspects, the external sensor can be selected from the group consisting of a humidity sensor, an ambient temperature sensor, a weight sensor, and a sensor disposed on a charging device for the ear-wearable dehydration monitoring system.

In a seventy-third aspect, in addition to one or more of the preceding or following aspects, or in the alternative to some aspects, the ear-wearable dehydration monitoring system can be configured to process signals of the microphone to detect signs of dehydration.

In a seventy-fourth aspect, in addition to one or more of the preceding or following aspects, or in the alternative to some aspects, the signs of dehydration include smacking or licking lips.

In a seventy-fifth aspect, in addition to one or more of the preceding or following aspects, or in the alternative to some aspects, the signs of dehydration include pitch tremor.

In a seventy-sixth aspect, in addition to one or more of the preceding or following aspects, or in the alternative to some aspects, the signs of dehydration include rapid shallow breathing.

In a seventy-seventh aspect, in addition to one or more of the preceding or following aspects, or in the alternative to some aspects, the signs of dehydration include dysphonia.

In a seventy-eighth aspect, in addition to one or more of the preceding or following aspects, or in the alternative to some aspects, the ear-wearable dehydration monitoring system can be configured to issue an alert when dehydration clinical symptoms cross a threshold value.

In a seventy-ninth aspect, in addition to one or more of the preceding or following aspects, or in the alternative to some aspects, the threshold value can be dynamically set based on factors including one or more of an ambient temperature, an ambient humidity, and activity levels of the device wearer.

In an eightieth aspect, in addition to one or more of the preceding or following aspects, or in the alternative to some aspects, the ear-wearable dehydration monitoring system can be configured to identify drinking events based at least in part on signals from the microphone and record the same.

In an eighty-first aspect, in addition to one or more of the preceding or following aspects, or in the alternative to some aspects, the ear-wearable dehydration monitoring system can be configured to issue an alert if a number of identified drinking events over a defined time period change by at least a threshold value.

In an eighty-second aspect, in addition to one or more of the preceding or following aspects, or in the alternative to some aspects, the ear-wearable dehydration monitoring system can be configured to issue an alert when dehydration clinical symptoms cross a threshold value for at least a threshold period of time.

In an eighty-third aspect, in addition to one or more of the preceding or following aspects, or in the alternative to some aspects, the ear-wearable dehydration monitoring system can further include a first unit, wherein the first unit can be configured to be wearable about a first ear, and a second unit, wherein the second unit can be configured to be wearable about a second ear, wherein signals can be exchanged between the first unit and the second unit.

In an eighty-fourth aspect, in addition to one or more of the preceding or following aspects, or in the alternative to some aspects, the ear-wearable dehydration monitoring system can be configured to classify an observed pattern representing signals from the microphone and the sensor package into a scale of dehydration severities using a machine learning derived algorithm.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of an ear-wearable hydration level monitoring device and a device wearer in accordance with various embodiments herein.

FIG. 2 is a schematic view of fluid inputs and outputs of a device wearer in accordance with various embodiments herein.

FIG. 3 is a schematic view of an ear-wearable hydration level monitoring device in accordance with various embodiments herein.

FIG. 4 is a schematic view of an ear-wearable hydration level monitoring device within an ear of a device wearer in accordance with various embodiments herein.

FIG. 5 is a block diagram illustrating sensors and hydration level symptoms in accordance with various embodiments herein.

FIG. 6 is a block diagram illustrating sensors and hydration level symptoms in accordance with various embodiments herein.

FIG. 7 is a schematic view of an accessory device in accordance with various embodiments herein.

FIG. 8 is a schematic view of an ear-wearable hydration level monitoring system in accordance with various embodiments herein.

FIG. 9 is a schematic view of an ear-wearable hydration level monitoring system in accordance with various embodiments herein.

FIG. 10 is a schematic view of an ear-wearable hydration level monitoring device within an ear of a device wearer in accordance with various embodiments herein.

FIG. 11 is a schematic view of an ear-wearable hydration level monitoring device within an ear of a device wearer in accordance with various embodiments herein.

FIG. 12 is a schematic view of a humidity shroud in accordance with various embodiments herein.

FIG. 13 is a schematic view of an ear-wearable hydration level monitoring device in accordance with various embodiments herein.

FIG. 14 is a schematic view of an ear-wearable hydration level monitoring device within an ear of a device wearer in accordance with various embodiments herein.

FIG. 15 is a schematic view of hydration level, fluid consumption, and hydration level symptom severity over time.

FIG. 16 is a block diagram of components of an ear-wearable hydration level monitoring device in accordance with various embodiments herein.

FIG. 17 is a block diagram of components of an accessory device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, dehydration represents a significant health issue. This is particularly true among elderly people as they are physiologically more susceptible due to changes in bodily fluid reserves, renal function, and thirst response.

In the absence of a gold standard for diagnosing dehydration, observing clinical symptoms of hydration level is a key approach to detection. Systems and devices herein can automatically assess hydration to detect dehydration and notify the individual experiencing dehydration and/or a caregiver. This monitoring technology can provide benefits for users across all age groups. However, this monitoring technology can provide particular benefits for the elderly including prolonging how long elderly individuals are able to live independently and/or improving the quality of care received by individuals residing in assisted living.

Systems herein can include an ear-wearable device including a receiver and sensor package containing various sensors, such as microphone, photoplethysmography (PPG) sensor, electrocardiography (ECG) sensor, temperature sensor, electromyography (EMG) sensor, inertial measurement unit (IMU) sensor, electroencephalography (EEG) sensor, and glucose sensor. These sensors can be used individually or in conjunction to detect symptoms of hydration level or dehydration (including, but not limited to, increased pulse, rapid/shallow breathing, low blood pressure, dizziness, dry mouth, change in voice quality, increased temperature, delirium, and increased glucose concentration). For example, rapid, shallow breathing may be detected acoustically via microphone or by monitoring respiratory sinus arrhythmia via ECG or PPG. A single sensor can be used to detect multiple symptoms. For example, the microphone can detect a change in vocal quality in addition to detecting rapid, shallow breathing.

Aspects of embodiments herein can include the application of voice quality and core body temperature analysis to the detection of hydration level. Audio input to detect change in voice and the temperature increase are two elements that can be tied directly to the hydration level condition leading to improved sensitivity and specificity of hydration level detection. Combining voice quality and temperature data with the data output of sensors such as optical sensors and motion sensors can provide a rich set of biometric data leading to robust hydration level detection sufficient to allow discrimination between dehydration and other conditions that could otherwise appear similar to dehydration when only using a more limited set of biometric data.

Additional aspects of embodiments herein can include long-term tracking and personalized hydration level determination on an ear-wearable platform. Personalized baselines can be established to more accurately differentiate abnormal conditions. For example, a resting heart rate of 70 bpm may be clinically considered as normal, but for a trained athlete with normal resting heart rate of 50 bpm, a 70 bpm HR should be considered as elevated. In the case of blood pressure, many hypertensive patients may go through their day with systolic pressure above 140 mmHg even well hydrated. Therefore, a universal absolute threshold for detecting hydration level symptoms is unlikely to work across population. With an ear-wearable device that is worn every day over long periods of time, individualized baselines can be established and provide more accurate and personalized hydration level alerts.

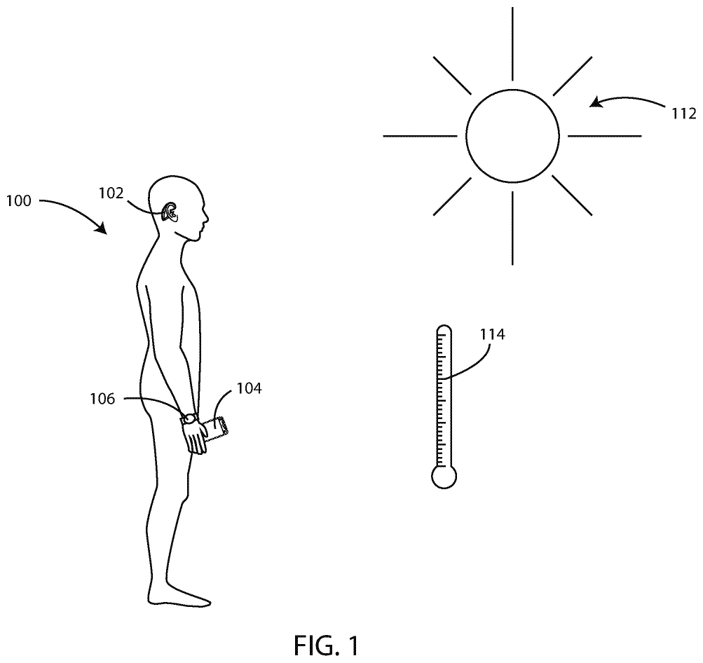

Referring now to FIG. 1, a schematic view of an ear-wearable hydration level monitoring device 102 and a device wearer 100 is shown in accordance with various embodiments herein. The weather 112, and specifically the temperature 114 and humidity, experienced by the device wearer 100 will impact water loss through perspiration. This can occur even if the device wearer 100 is substantially sedentary. Thus, environmental conditions (outdoors or indoors) can play a role in the development of dehydration. In this view, the device wearer 100 is shown wearing an ear-wearable hydration level monitoring device 102. In accordance with embodiments herein, the ear-wearable hydration level monitoring device 102 can be used to monitor wearers of the system/device for possible dehydration. In some embodiments, the ear-wearable monitoring device 102 can also work in combination with other devices. For example, in FIG. 1 the device wearer 100 is also depicted with an accessory device 104 and a separate wearable device 106. In some embodiments, these other devices can be used to provide additional data (such as based on sensors that are part of the other devices) for analysis and/or can used to convey alerts or information regarding the possible detection of dehydration.

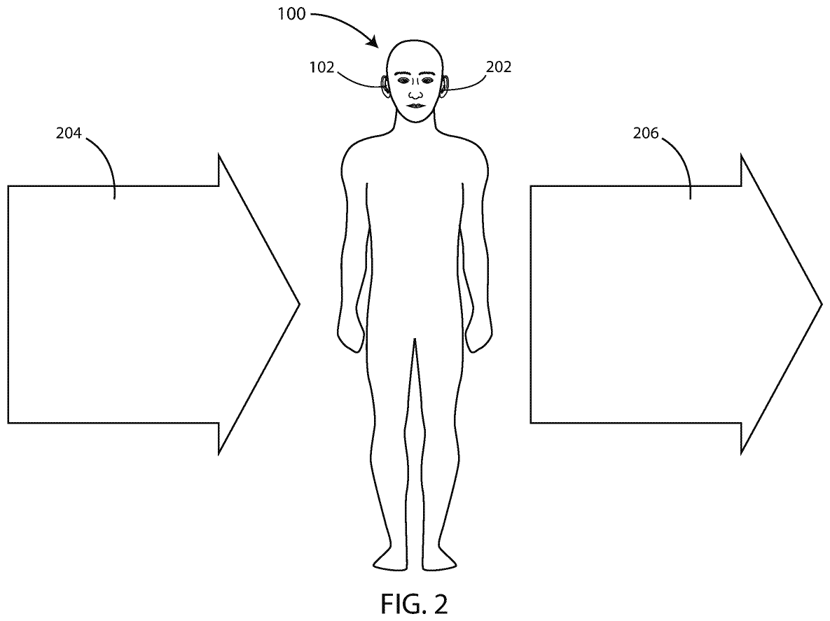

Referring now to FIG. 2, a schematic view of fluid inputs and outputs is shown as they might impact a device wearer 100. Fluid (water) inputs 204 can include consumption of fluids (water, beverages, etc.) as well as eating foods and thereby obtaining the moisture content of such foods. Sources of water can also include a portion of water produced by metabolism. Fluid (water) outputs 206 can include water lost through urination, perspiration, evaporation from the lungs, emesis, passing feces, dysentery, and the like. Generally, the human body has approximately 60 percent water. Loss of water resulting in weight loss of less than 5% can be deemed mild dehydration. Loss of water resulting in weight loss of from 5% to 9% can be deemed moderate dehydration. Loss of water resulting in weight loss of from 10% or more can be deemed severe dehydration.

As before, the device wearer 100 can be monitored using an ear-wearable hydration level monitoring system that can specifically include an ear-wearable device 102. In various scenarios, the ear-wearable hydration level monitoring system can also include a second ear-wearable device 202 that is worn on or about the other ear of the device wearer 100. In some embodiments, the ear-wearable devices 102 and 202 can function substantially independently and monitor for hydration level redundantly. In other embodiments, the ear-wearable device 102 can exchange signals/data and function cooperatively to more accurately detect possible dehydration.

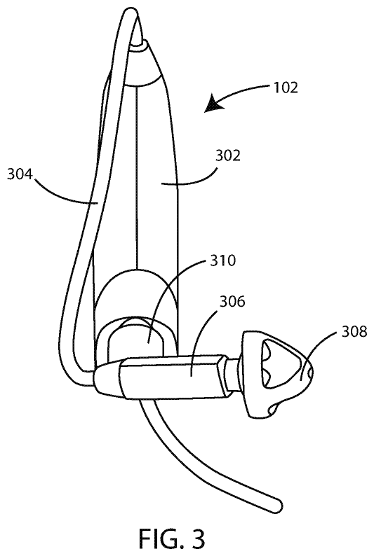

Ear-wearable devices herein can take on many different specific forms. Referring now to FIG. 3, a schematic view of an example of an ear-wearable hydration level monitoring device 102 is shown in accordance with various embodiments herein. The ear-wearable monitoring device 102 can include a housing 302, a cable 304, a receiver 306, a cone 308, and a battery compartment 310, amongst other things.

In various embodiments, the ear-wearable monitoring device 102 can include a control circuit, a microphone in electrical communication with the control circuit, a power supply in electrical communication with the control circuit, and a sensor package. The sensor package can include various sensors as described further below. The ear-wearable hydration level monitoring device 102 can be configured to process signals of one or more sensors of the sensor package to detect various clinical symptoms of hydration level or dehydration. In various embodiments, the ear-wearable hydration level monitoring device 102 can also be configured to process signals of the microphone to detect signs of hydration level. In various embodiments, the ear-wearable hydration level monitoring device 102 can be configured to issue an alert when hydration level or dehydration symptoms cross a threshold value and are sustained for at least a threshold period of time.

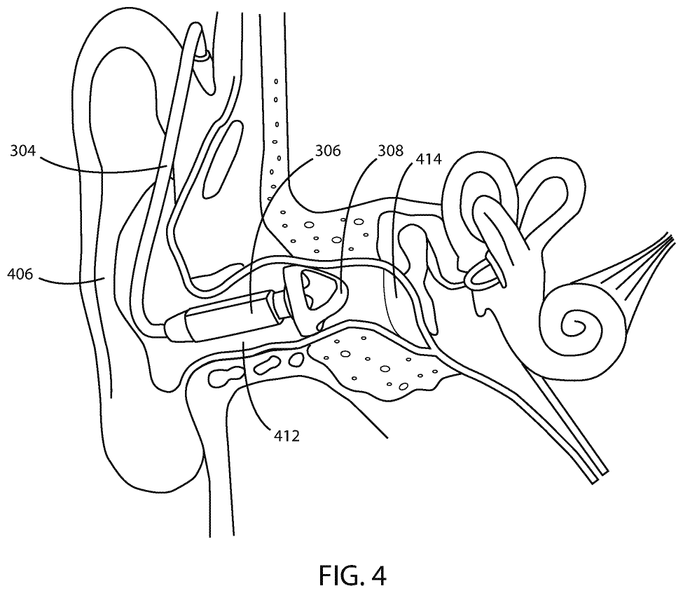

Ear-wearable devices herein can be worn on or in the ear. For example, referring now to FIG. 4, a schematic view of an ear-wearable hydration level monitoring device is shown within an ear of a device wearer 100 in accordance with various embodiments herein. FIG. 4 shows the external ear 406, the external auditory canal 412 and the tympanic membrane 414. Portions of the ear-wearable device are visible including a cable 304 connecting to a receiver 306 and a cone 308 disposed on the end thereof.

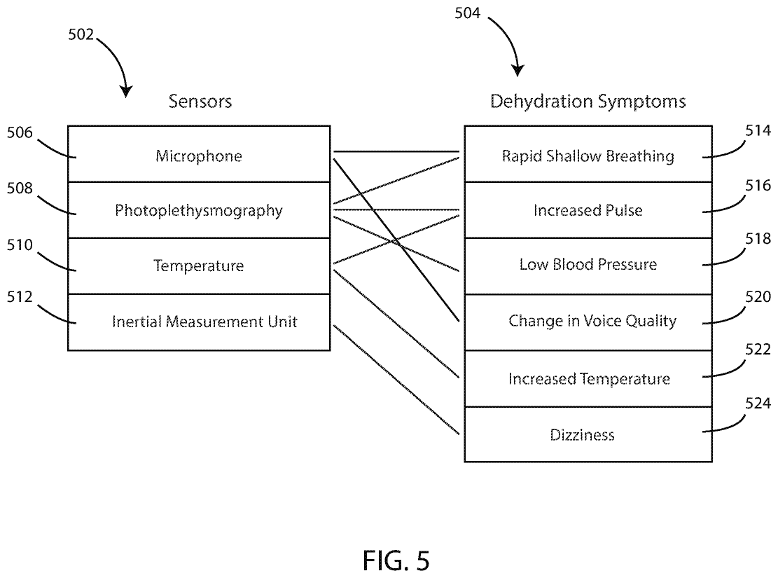

It will be appreciated that many different sensors can be included with embodiments herein and can detect various clinical symptoms of hydration level or dehydration. Referring now to FIG. 5, a block diagram illustrating sensors and dehydration symptoms is shown in accordance with various embodiments herein. In specific, FIG. 5 shows some exemplary sensors of a sensor package 502. The sensor package 502 can be configured to include various sets of sensors, depending on the specific embodiment. Thus, a sensor package 502 in accordance with embodiments herein can include one, two, three, or four of the sensors shown in FIG. 5.

In this particular example, the sensor package 502 includes a photoplethysmography sensor 508, a temperature sensor 510, a motion sensor 512, and a microphone 506. FIG. 5 also shows clinical symptoms of dehydration 504. The clinical symptoms of dehydration 504 can include a rapid shallow breathing 514, an increased pulse rate 516, a low blood pressure 518, a change in voice quality 520, an increased temperature 522, dizziness 524, and the like.

While a normal breathing rate will be different for each individual, breathing rates amongst an elderly set of patients can be from 12 to 18 breaths per minute for those living independently and 16 to 25 breaths per minute for those in long term-care. Further, while a normal pulse rate can depend on various factors, a normal pulse rate amongst the elderly can be from 60 to 100 beats per minute. Similarly, while a normal blood pressure for an individual can depend on various factors, a normal blood pressure is generally less than 120/80.

However, for most of the clinical symptoms of dehydration, it is particularly valuable to understand what normal values are for a given patient. As such, embodiments herein can detect such values and establish a baseline value for an individual over a period of time. For example, systems and/or devices herein can monitor data over time periods of hours, days, weeks, months or years in order to derive a baseline value for any of the measures that can serve as signs of dehydration. In some embodiments, the baseline value can be a moving average value of any of the measures or any combination thereof. In some embodiments, the baseline value can be a statistical measure. In some embodiments, the baseline value can account for diurnal cycles. For example, blood pressure typically rises sharply on waking in the morning and falls during sleep at night.

Thus, in various embodiments herein, symptoms of dehydration can include one or more of in increased rate of breathing over a baseline value, an increased pulse rate over a baseline value, a decreased blood pressure over a baseline value, an increased temperature over a baseline value, and/or an increase in dizziness or unsteadiness over a baseline value.

Changes in voice quality can also be a sign of dehydration. Voice quality can be assessed by capturing an individual's voice using a microphone. The signals from the microphone can then be processed using analog and/or digital signal processing techniques. As such, in various embodiments herein, an ear-wearable device can be configured to detect signs of dehydration including changes in voice pitch (typically a lowered pitch/frequency associated with hoarseness) and tremor (e.g., a quavering of the voice). In various embodiments herein, an ear-wearable device can be configured to detect signs of dehydration including dysphonia (hoarseness).

In various embodiments, the ear-wearable device or system can distinguish between speech or sounds associated with the device wearer and speech or sounds associated with a third party. This can be useful to be sure that detected changes in voice quality actually relate to the device wearer instead of another nearby individual.

Distinguishing between speech or sounds associated with the device wearer and speech or sounds associated with a third party can be performed in various ways. In some embodiments, this can be performed through signal analysis of the signals generated from the microphone(s). For example, in some embodiments, this can be done by filtering out frequencies of sound that are not associated with speech of the device-wearer. In some embodiments, such as where there are two or more microphones (on the same ear-wearable device or on different ear-wearable devices) this can be done through spatial localization of the origin of the speech or other sounds and filtering out, spectrally subtracting, or otherwise discarding sounds that do not have an origin within the device wearer. In some embodiments, such as where there are two or more ear-worn devices, own-voice detection can be performed and/or enhanced through correlation or matching of intensity levels and or timing.

In some cases, the system can include a bone conduction microphone in order to preferentially pickup the voice of the device wearer. In some cases, the system can include a directional microphone that is configured to preferentially pickup the voice of the device wearer. In some cases the system can include an intracanal microphone (a microphone configured to be disposed within the ear-canal of the device wearer) to preferentially pickup the voice of the device wearer. In some cases, the system can include a motion sensor (e.g., an accelerometer configured to be on or about the head of the wearer) to preferentially pick up skull vibrations associated with the vocal productions of the device wearer.

In some cases, an adaptive filtering approach can be used. By way of example, a desired signal for an adaptive filter can be taken from a first microphone and the input signal to the adaptive filter is taken from the second microphone. If the hearing aid wearer is talking, the adaptive filter models the relative transfer function between the microphones. Own-voice detection can be performed by comparing the power of an error signal produced by the adaptive filter to the power of the signal from the standard microphone and/or looking at the peak strength in the impulse response of the filter. The amplitude of the impulse response should be in a certain range in order to be valid for the own voice. If the user's own voice is present, the power of the error signal will be much less than the power of the signal from the standard microphone, and the impulse response has a strong peak with an amplitude above a threshold. In the presence of the user's own voice, the largest coefficient of the adaptive filter is expected to be within a particular range. Sound from other noise sources results in a smaller difference between the power of the error signal and the power of the signal from the standard microphone, and a small impulse response of the filter with no distinctive peak. Further aspects of this approach are described in U.S. Pat. No. 9,219,964, the content of which is herein incorporated by reference.

In another approach, a system herein can use a set of signals from a number of microphones. For example, a first microphone can produce a first output signal A from a filter and a second microphone can produce a second output signal B from a filter. The apparatus includes a first directional filter adapted to receive the first output signal A and produce a first directional output signal. A digital signal processor is adapted to receive signals representative of the sounds from the user's mouth from at least one or more of the first and second microphones and to detect at least an average fundamental frequency of voice (pitch output) F₀. A voice detection circuit is adapted to receive the second output signal B and the pitch output F₀ and to produce an own voice detection trigger T. The apparatus further includes a mismatch filter adapted to receive and process the second output signal B, the own voice detection trigger T, and an error signal E, where the error signal E is a difference between the first output signal A and an output O of the mismatch filter. A second directional filter is adapted to receive the matched output O and produce a second directional output signal. A first summing circuit is adapted to receive the first directional output signal and the second directional output signal and to provide a summed directional output signal (D). In use, at least the first microphone and the second microphone are in relatively constant spatial position with respect to the user's mouth, according to various embodiments. Further aspects of this approach are described in U.S. Pat. No. 9,210,518, the content of which is herein incorporated by reference.

In various embodiments, the ear-wearable hydration level monitoring system (described further below) can be configured to classify an observed pattern representing signals from the microphone 506 and the sensor package 502 into a scale of hydration level or dehydration severities using a machine learning derived algorithm. By way of example, dehydration can be classified into mild dehydration, moderate dehydration, and severe dehydration. In some embodiments, machine learning analysis (such as the use of a machine learning classification algorithm) can be used to evaluate current clinical measures of dehydration (including any of those mentioned herein) and classify the same as being evidence of mild dehydration, moderate dehydration, and severe dehydration.

In various embodiments herein, one or more sensors can be operatively connected to a controller (such as a control circuit described further below) or another processing resource (such as a processor of another device or a processing resource in the cloud). The controller or other processing resource can be adapted to receive data representative of a characteristic of the subject from one or more of the sensors and/or determine statistics of the subject over a monitoring time period based upon the data received from the sensor. As used herein, the term “data” can include a single datum or a plurality of data values or statistics. The term “statistics” can include any appropriate mathematical calculation or metric relative to data interpretation, e.g., probability, confidence interval, distribution, range, or the like. Further, as used herein, the term “monitoring time period” means a period of time over which characteristics of the subject are measured and statistics are determined. The monitoring time period can be any suitable length of time, e.g., 1 millisecond, 1 second, 10 seconds, 30 seconds, 1 minute, 10 minutes, 30 minutes, 1 hour, 1 day, 1 week, etc., or a range of time between any of the foregoing time periods.

Any suitable technique or techniques can be utilized to determine statistics for the various data from the sensors, e.g., direct statistical analyses of time series data from the sensors, differential statistics, comparisons to baseline or statistical models of similar data, etc. Such techniques can be general or individual-specific and represent long-term or short-term behavior. These techniques could include standard pattern classification methods such as Gaussian mixture models, clustering as well as Bayesian approaches, neural network models and deep learning.

Further, in some embodiments, the controller can be adapted to compare data, data features, and/or statistics against various other patterns, which could be prerecorded patterns (baseline patterns) of the particular individual wearing an ear-wearable device herein, prerecorded patterns (group baseline patterns) of a group of individuals wearing ear-wearable devices herein, one or more predetermined patterns that serve as patterns indicative of particular hydration levels or dehydration (positive example patterns), one or more predetermined patterns that service as patterns indicative of the absence of particular hydration levels or dehydration (negative example patterns), or the like. As merely one scenario, if a pattern is detected in an individual that exhibits similarity crossing a threshold value to a positive example pattern or substantial similarity to that pattern, then that can be taken as an indication of an occurrence of a particular hydration level or dehydration.

Similarity and dissimilarity can be measured directly via standard statistical metrics such normalized Z-score, or similar multidimensional distance measures (e.g. Mahalanobis or Bhattacharyya distance metrics), or through similarities of modeled data and machine learning. These techniques can include standard pattern classification methods such as Gaussian mixture models, clustering as well as Bayesian approaches, neural network models, and deep learning.

As used herein the term “substantially similar” means that, upon comparison, the sensor data are congruent or have statistics fitting the same statistical model, each with an acceptable degree of confidence. The threshold for the acceptability of a confidence statistic may vary depending upon the subject, sensor, sensor arrangement, type of data, context, condition, etc.

The statistics associated with the health status of an individual (and, in particular, their status with respect to hydration level), over the monitoring time period, can be determined by utilizing any suitable technique or techniques, e.g., standard pattern classification methods such as Gaussian mixture models, clustering, hidden Markov models, as well as Bayesian approaches, neural network models, and deep learning.

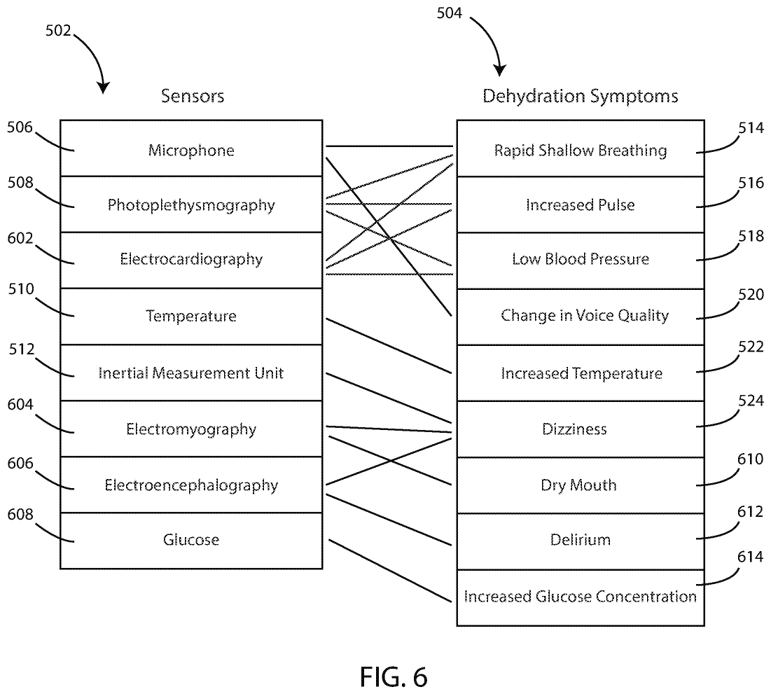

In some embodiments, the system can include and/or utilize a greater or lesser number of sensors. For example, referring now to FIG. 6, a block diagram is shown illustrating sensors and dehydration symptoms in accordance with other embodiments herein. As before, the ear-wearable device includes a sensor package 502. However, in this example, the sensor package 502 includes a photoplethysmography sensor 508, a temperature sensor 510, a motion sensor 512, an electrocardiography sensor 602, an electromyography sensor 604, an electroencephalography sensor 606, a glucose sensor 608, and a microphone 506. By utilizing a greater set of sensors, an even larger range of

clinical symptoms of dehydration 504 can be detected. In this embodiments, clinical symptoms of dehydration 504 can include rapid shallow breathing 514, an increased pulse rate 516, a low blood pressure 518, a change in voice quality 520, an increased temperature 522, dizziness 524, a dry mouth 610, delirium 612, and an increased glucose concentration 614.

Beyond changes in voice quality, microphones herein can be used to detect other occurrences that can be indicative of hydration levels or dehydration. By way of example, in various embodiments, the signs of dehydration include can include smacking or licking lips. The smacking or licking of lips result in unique aural signatures/patterns that can be detected by evaluating the signals from a microphone herein using analog and/or digital signal processing techniques and techniques such as pattern matching approaches described in greater detail below.

In various embodiments, the ear-wearable dehydration monitoring system can be configured to issue an alert, notification, or warning when dehydration clinical symptoms cross a threshold value. Alerts, notifications, and warnings can take various forms including audio notifications such as warning sounds or warning messages delivered by the ear-wearable device or another device, visual notifications such as notification messages on an accessory device, network delivered notifications, haptic notifications, and the like.

High ambient temperature (such as above 75, 80, 85, or 90 degrees Fahrenheit), high humidity (such as above 80, 85, or 90 percent relative humidity), and high or above average activity of the device wearer can all created conditions where water loss can be particularly rapid. As such, in some embodiments, the risk of dehydration can be particularly acute when high ambient temperature, high humidity, and/or high activity levels are detected. The risk of dehydration can also be high in very low humidity environments. In response to such elevated risk, in various embodiments herein, a threshold value for issuing an alert, notification, or warning can be dynamically set based on factors including one or more of an ambient temperature 114, an ambient humidity, and activity levels of the device wearer 100. In various embodiments, the threshold value can be lowered (e.g., the sensitivity of detection can be increased) under such conditions so that an alert can be sent sufficiently early to mitigate the onset and effects of dehydration.

In some embodiments, a system herein can also include and/or utilize an accessory device. The accessory device can be used for various purposes. In some embodiments, the accessory device can be used to provide data from additional sensors that may be a part of the accessory device.

In various embodiments herein, the ear-wearable dehydration monitoring system can be configured to receive data from at least one external sensor or external source. In various embodiments herein, the external sensor can be including at least one of a humidity sensor, an ambient temperature sensor, a weight sensor, and a sensor disposed on a charging device for the ear-wearable dehydration monitoring system. External sources can include, for example, things such as a source of weather information (such as a weather API), an electronic medical record, or the like.

In some embodiments, an accessory device (such as a smart phone) can be used to provide instructions or recommendations to the device wearer, such as instructions for mitigating a detected state of dehydration. In some embodiments, the accessory device can be used to provide instructions or recommendations to a care provider or health professional, such as instructions for mitigating a detected state of dehydration.

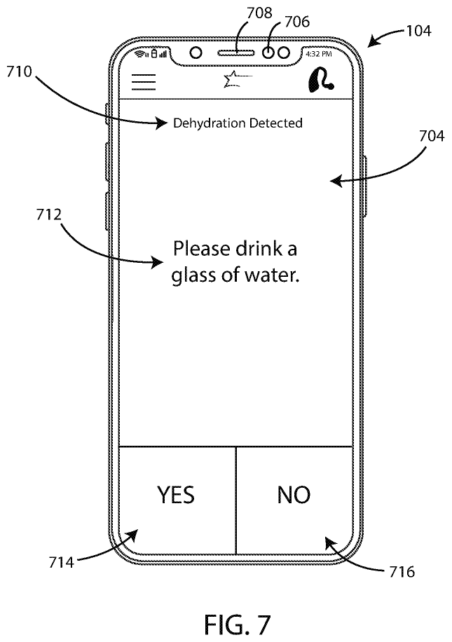

Referring now to FIG. 7, a schematic view of an accessory device 104 is shown in accordance with various embodiments herein. The accessory device 104 includes a display screen 704. The accessory device 104 also includes a camera 706 and a speaker 708. The accessory device 104 also shows a notification 710. In this case, the notification 710 states that a state of dehydration has been detected. The accessory device 104 also includes a suggested action 712. In this case, the suggested action 712 is to drink a glass of water. However, other suggested actions can include, but are not limited to, seeking assistance, returning to a cooler environment, getting out of the sun, and the like. In some embodiments, the accessory device 104 can display a query for the device wearer or a message that otherwise solicits or offers a chance for user input. For example, the system can seek confirmation of a likely instance of dehydration by querying the device wearer with a question such as “is your throat dry”? As such, in some embodiments, the accessory device 104 also includes a first user input element 714 and/or a second user input element 716 by which user input can be received. However, it will be appreciated that user input can also be provided in other ways such as by receiving spoken commands from the device wearer or another individual.

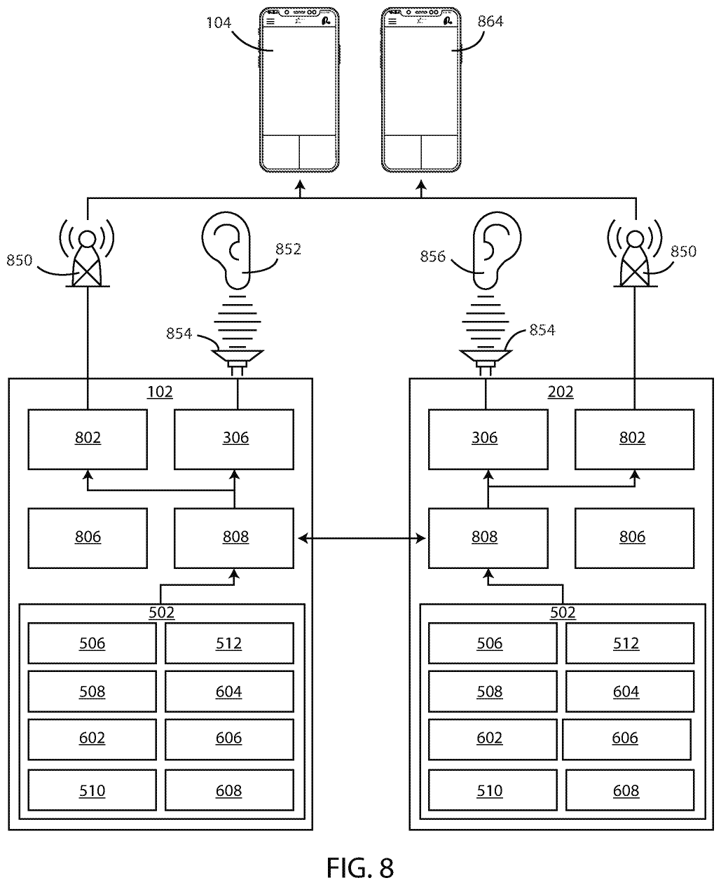

Ear-wearable hydration level monitoring devices and/or systems herein can include many different components. Referring now to FIG. 8, a schematic view of an ear-wearable hydration level monitoring system is shown in accordance with various embodiments herein. In this embodiment, the system includes a first ear-wearable hydration level device 102 and a second ear-wearable hydration level device 202. The first ear-wearable hydration level device 102 and be disposed on or within a first ear 852 and the second ear-wearable hydration level device 202 can be disposed on or within a second ear 856.

The ear-wearable devices 102, 202 can include various components such as a receiver 306 and a microphone 506. The ear-wearable devices 102, 202 can also include a data store containing a data log 802. The ear-wearable devices 102, 202 also includes a battery 806. The ear-wearable devices 102, 202 also includes a machine learning processing unit 808. The machine learning processing unit can include components such as those described with respect to a control circuit herein. The machine learning processing unit can function to execute machine learning algorithms on data provided by the sensors and/or utilize patterns and/or algorithms derived using machine learning analysis with respect to the sensor data. The ear-wearable devices 102, 202 can also include an antenna 850 and an electroacoustic transducer 854.

The ear-wearable devices 102, 202 include a sensor package 502 that can include a photoplethysmography sensor 508, a temperature sensor 510, a motion sensor 512, an electrocardiography sensor 602, an electromyography sensor 604, an electroencephalography sensor 606, and/or a glucose sensor 608. In this embodiment, the ear-wearable hydration level monitoring system also includes a first accessory device 104 (such as a smartphone or other computing device) and a second accessory device 864.

Once a hydration level or dehydration is detected, the system can notify the device wearer by relaying a notification acoustically via receiver in the ear or electronically to an accessory or monitoring device with wireless capability (e.g., smartphone, smartwatch, tablet, computer, etc.). Electronic notification can be transmitted to the device wearer's accessory device or a monitoring device of a personal (e.g., family member) or professional (e.g., assisted living staff) caretaker.

In various embodiments, the ear-wearable hydration level monitoring system (described further below) can be configured to classify an observed pattern representing signals from the microphone 506 and the sensor package 502 into a scale of hydration levels or dehydration severities using a machine learning derived algorithm.

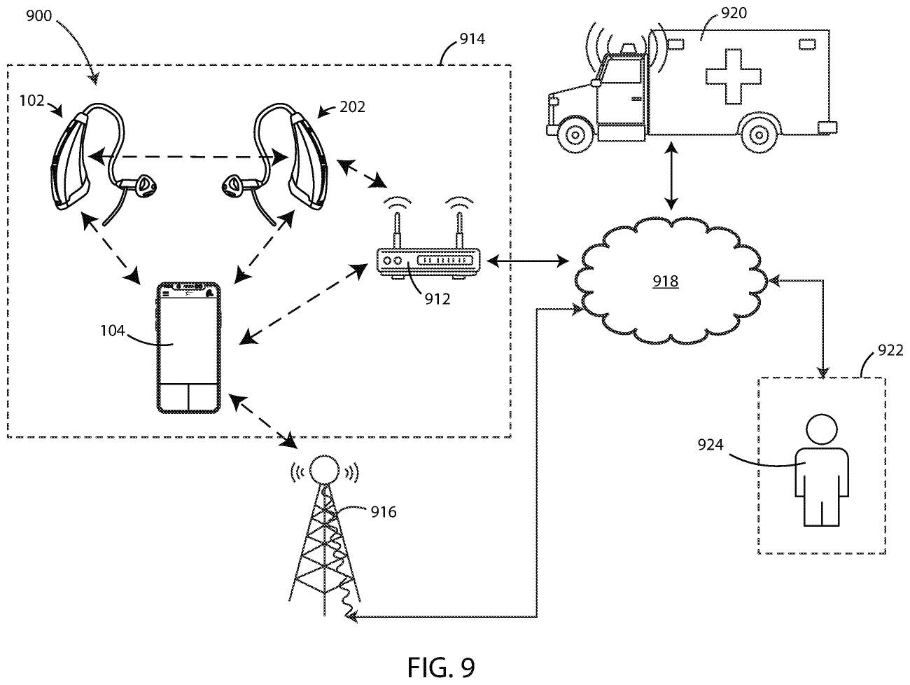

Referring now to FIG. 9, a schematic view of an ear-wearable hydration level monitoring system 900 is shown in accordance with various embodiments herein. The ear-wearable hydration level monitoring system 900 can include a first ear-wearable device 102, an accessory device 104, and a second ear-wearable device 202. In various embodiments, the first ear-wearable device 102, an accessory device 104, and a second ear-wearable device 202 can all be at a first location 914 where the device wearer is located. Signals from various components of the system and/or notifications can be conveyed remotely such as to and/or through the cloud 918. For example, the first location 914 can include a network router 912 that can serve as a gateway for network communication. FIG. 9 also shows a cell tower 916 can be used to exchange signals with the accessory device 104 and/or the ear-wearable devices. Signals and/or notifications conveyed remotely can be directed to different parties. FIG. 9 shows a second location 922 and a caretaker 924 at the second location 922. Commonly, notifications herein can be directed to the caretaker 924 whether the caretaker 924 is at the first location 914 or the second location 922. However, in some embodiments, the notifications may be directed so as to receive a possibly more timely and serious response. For example, FIG. 9 shows an emergency responder 920. In cases of extreme dehydration, a notification can be sent directly to the emergency responder 920 to request their assistance.

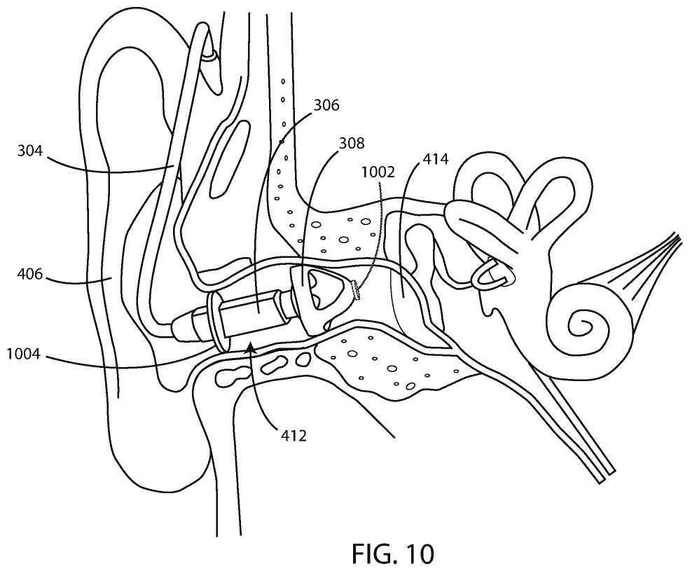

In some embodiments, hydration levels or dehydration can be detected by sensing the humidity of an air-filled area of the body, such as within the external auditory ear canal. Referring now to FIG. 10, a schematic view of an ear-wearable hydration level monitoring device within an ear of a device wearer is shown in accordance with various embodiments herein. As with some of the previous figures, the external ear 406 is shown along with the external auditory canal 412 and the tympanic membrane 414. Components of an ear-wearable device are shown including a cable 304, a receiver 306, and a cone 308. The ear-wearable device can include a humidity sensor 1002 in order to detect the relative humidity within the external auditory canal or a portion thereof. While not intending to be bound by theory, it is believe that accurate measurements of humidity within the ear canal that can be indicative of hydration levels or dehydration are facilitated by at least partially blocking off a portion of the external ear-canal so that humidity therein does not simply pass through the external auditory canal and out the external ear to dissipate in the surrounding environment. As such, in some embodiments the ear-wearable device includes a sealing member 1004. The sealing member 1004 can take on various forms. In some embodiments, the sealing member 1004 can take the form of a sealing baffle and can be mounted on another component of the ear-wearable device such as mounted on the receiver 306 as shown in FIG. 10. In some embodiments, the sealing member 1004 can take the form of a sealing dome. In this case, the sealing member 1004 isolates a portion of the external auditory canal adjacent the tympanic membrane. In various embodiments the sealing member can be attached to a structure (such as a device housing, a receiver, or another structure) and a humidity sensor can be attached to the same structure. However, in other embodiments the sealing member and the humidity sensor can be attached to different structures. In some embodiments herein, the humidity sensor 1002 can be configured to measure humidity within an ear canal of a wearer of the ear-wearable hydration level monitoring system 900 between the sealing dome and a tympanic membrane 414 of the wearer.

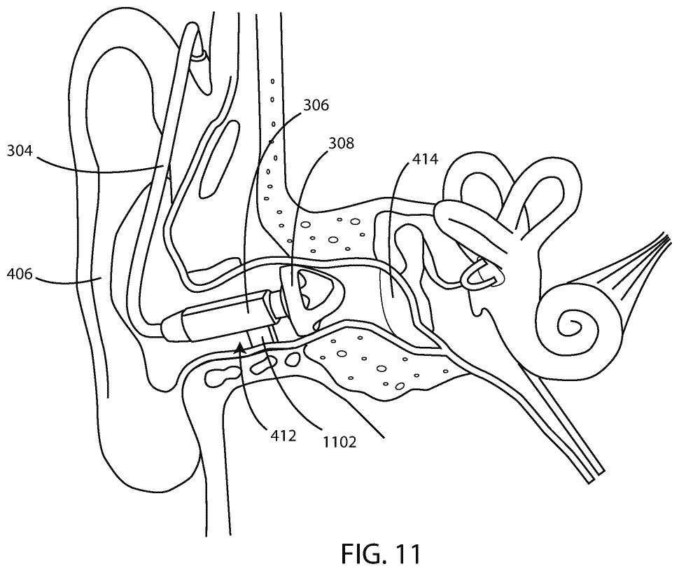

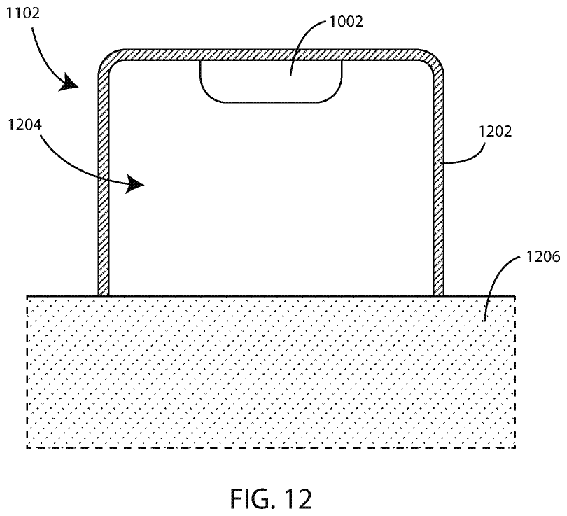

In some embodiments, a shroud or similar cover can be used around the hydration sensor to isolate a small space within the external auditory canal. Referring now to FIG. 11, a schematic view of an ear-wearable hydration level monitoring device within an ear of a device wearer 100 is shown in accordance with various embodiments herein. FIG. 11 is generally similar to FIG. 10. However, in this example, FIG. 10 depicts a device without the sealing member of FIG. 10, but including a humidity shroud 1102. FIG. 12 shows a schematic sectional view of a humidity shroud 1102 in accordance with various embodiments herein. The external auditory canal has an auditory canal surface 1206. The humidity shroud 1102 includes a shroud housing 1202 defining an interior space 1204. A humidity sensor 1002 can disposed so that it can measure humidity within the interior space 1204.

It will be appreciated that ear-wearable devices herein can take on many different forms. In some embodiments, the ear-wearable device can be in the form of an in-the-ear style custom ear-wearable device. While not intending to be bound by theory, it is believed that certain form factors, such as an in-the-ear style custom ear-wearable device, can have better mechanical coupling to the external auditory canal which can be advantageous for measuring humidity therein.

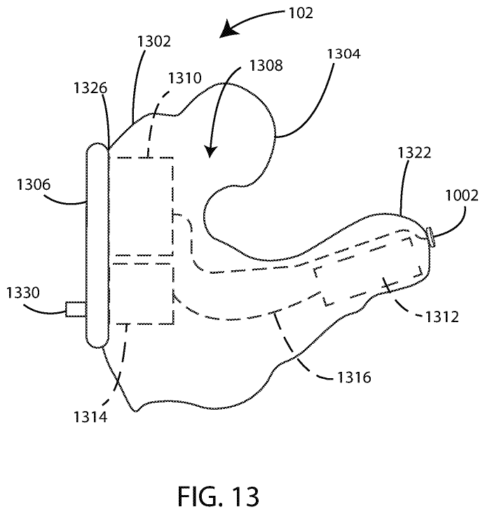

Referring now to FIG. 13, a schematic view of an in-the-ear style custom ear-wearable device 102 which can be used as a hydration level monitoring device herein is shown in accordance with various embodiments herein. The ear-wearable device 102 can include an ear-wearable device housing 1302 formed by a shell 1304 and a faceplate 1306. The shell 1304 can be custom shaped to mate with the user's ear anatomy and can define an internal shell cavity 1308 and a shell aperture at the entrance to the shell cavity 1308. The faceplate 1306 is attached to the shell at the shell aperture to enclose the shell cavity 1308.

The ear-wearable device housing 1302 can define a battery compartment 1310 in which a battery can be disposed to provide power to the device. The ear-wearable device 102 can also include a receiver 1312. The receiver 1312 can include a component that converts electrical impulses into sound, such as an electroacoustic transducer, speaker, or loudspeaker. The housing 1302 can also define a component compartment 1314 that can contain electrical and other components including but not limited to a microphone, a processor, memory, various sensors, one or more communication devices, power management circuitry, and a control circuit. A cable 1316 or connecting wire can include one or more electrical conductors and provide electrical communication between components inside of the component compartment 1314 and components inside of the receiver 1312.

The shell 1304 extends from an ear canal end 1322 to an aperture end 1326. At the aperture end 1326, the shell 1304 defines an aperture that is closed by the faceplate 1306. The faceplate 1306 is sealed to the shell 1304. The faceplate 1306 is shown in FIG. 13 only in a side view but can include many features and structures. A user input device 1330 is shown as part of the faceplate in FIG. 13, and can be a button, lever, switch, dial, or other input device. The faceplate 1306 may also include a battery door, a microphone opening, a pull handle, and other features.

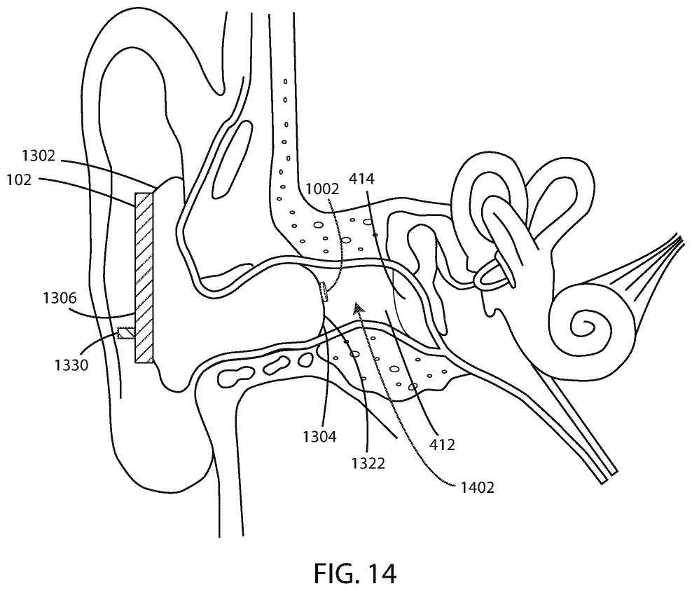

In various embodiments, a humidity sensor 1002 can be disposed on or adjacent to the ear canal end 1322. The humidity sensor 1002 can be, for example, a capacitive humidity sensor, a resistive humidity sensor, a thermal conductivity humidity sensor, or the like. When positioned within the ear canal (see, e.g., FIG. 14) the shell 1304 can act as a barrier to provide a space in in the ear canal in which humidity can be sensed more accurately. As such, positioning the humidity sensor 1002 on or adjacent the ear canal end 1322 of the shell 1304 provides an ideal location for the humidity sensor 1002 to sense humidity.

The ear-wearable device 102 shown in FIG. 13 is an in-the-ear style device and thus the shell is designed to be placed within the ear cavity. However, it will be appreciated that many different form factors for ear-wearable devices are contemplated herein. Aspects of ear-wearable devices and functions thereof are described in U.S. Pat. No. 9,848,273; U.S. Publ. Pat. Appl. No. 20180317837; and U.S. Publ. Pat. Appl. No. 20180343527, the content of all of which is herein incorporated by reference in their entirety.

FIG. 14 is a schematic view of an ear-wearable device 102 disposed within the ear of a wearer in accordance with various embodiments herein. The housing 1302 of the ear-wearable device 102 is defined by the shell 1304, which is positioned within the external auditory canal 412 (or ear canal), and the faceplate 1306, which is positioned in the concha. The user input device 1330 on the faceplate 1306 is accessible to be manipulated by the user without having to remove the ear-wearable device from their ear. The ear canal end 1322 of the shell 1304 is positioned close to the user's tympanic membrane. Ideally, the shell 1304 fits properly within the user's ear cavity. A proper fit is usually one in which the ear-wearable device forms an acoustic seal with the user's ear cavity, so that it is contacting the ear cavity around a circumference of the ear-wearable device at some location on the shell 1304 of the ear-wearable device 102. A proper fit is also comfortable to the user, so that the shell 1304 is not putting too much pressure on the walls of the external auditory canal 412 or features of the concha. The receiver 1312 (FIG. 13) is positioned within the shell 1304 at the ear canal end 1322 of the shell 1304 to minimize the distance between the receiver 1312 and the tympanic membrane 414 without physically contacting the tympanic membrane 414.

As can be seen in FIG. 14, the shell 1304 forms a barrier creating a space 1402 in in the ear canal in which humidity can be sensed more accurately. As such, positioning the humidity sensor 1002 on or adjacent the ear canal end 1322 of the shell 1304 allows the humidity sensor 1002 to be exposed to the space 1402 providing an ideal location for the humidity sensor 1002 to sense humidity.

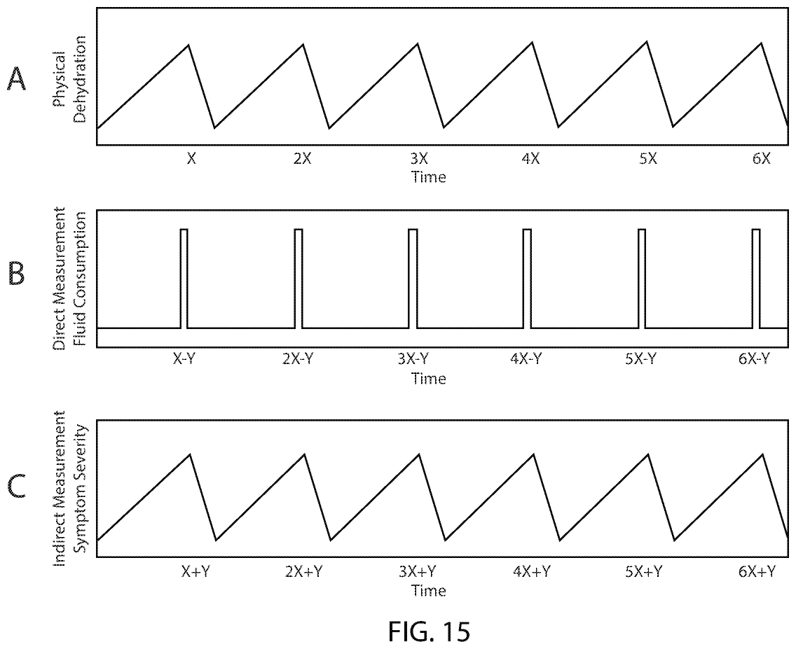

Referring now to FIG. 15, a schematic view of dehydration, fluid consumption, and dehydration symptom severity over time. With reference to chart “A”, the system can be configured to automatically track hydration over time “X” to determine when a user has successfully rehydrated, either directly by monitoring swallowing activity of the user (e.g., head tilt measured with IMU, swallowing activity measured by EMG, etc.) or indirectly by detecting the cessation of symptoms (e.g., decrease in heart measured with PPG or ECG). In the case of direct measurement, the fluid consumption will decrease dehydration at a negative time offset “Y” (see chart “B”), depending on the individuals' fluid absorption rate and quantity of fluids consumed. In the case of indirect measurement, symptom cessation will occur at a positive time offset “Z” (see chart “C”, depending on the device wearer's fluid absorption rate and symptom(s) observed.

In various embodiments, ear-wearable devices herein and related systems can be used to detect oropharyngeal events (both normal and abnormal) including, but not limited to, mastication, swallowing, drinking, and the like. These events can be used to identify events including the input of water (such as drinking of fluids). As such, embodiments herein include ear-worn devices and related systems that can be used to track aspects such as eating, drinking, swallowing, and other oropharyngeal events. In some embodiments, an exemplary a first ear-worn device can include a control circuit, a motion sensor, one or more microphones, an electroacoustic transducer, and a power supply or power supply circuit. The ear-worn device system can be configured to monitor signals from at least one of the motion sensor and the microphone and evaluate the signals to identify oropharyngeal events. As such, in various embodiments herein, an ear-wearable dehydration monitoring system can be configured to identify drinking events based at least in part on signals from the microphone and record the same.

Certain oropharyngeal events such as drinking are frequently accompanied by a characteristic head movement immediately prior to the event. For example, an individual commonly tips their head backward before beginning to drink from a glass. In some embodiments, ear-worn device systems herein are configured to evaluate the signals from a motion sensor to identify when the device wearer tips their head backward. In some embodiments, signal evaluation to identify oropharyngeal events includes evaluating signals from the motion sensor followed sequentially by evaluating signals from the microphone to detect sounds consistent with drinking.

In some embodiments, weighting factors for identification of oropharyngeal events, such as drinking events, can vary depending on whether another event is detected. For example, weighting factors can be changed such that signals from one or more microphones, motion sensors, or other sensors occurring immediately after head or jaw movement characteristic of the device wearer bringing a drink to their lips are more likely to be deemed a drinking event than are signals from the sensors in the absence of such head or jaw movements.

In various embodiments, devices and systems herein can be configured to distinguish between sounds originating at or near a sound origin associated with drinking versus sounds originating at other points within or outside of the body of the subject. In an embodiment, signal evaluation or processing to identify drinking events can include evaluating signals from the microphone of the first ear-worn device and signals from a microphone of the second ear-worn device and selecting those signals emanating from a spatial location that is laterally between the first ear-worn device and the second ear-worn device and posterior to the lips of the ear-worn device wearer.

In some embodiments, the number of identified drinking events (with or without an estimation of how much fluid was consumed) can be used to evaluate whether dehydration or circumstances that can lead to dehydration are present. In some embodiments, the system herein can track average numbers of drinking events over given time periods (such as per hour, per day, per week, etc.) and compare such numbers against those previously recorded for the individual (as one example of a baseline for the individual). In various embodiments, the ear-wearable dehydration monitoring system can be configured to issue an alert if a number of identified drinking events over a defined time period change by at least a threshold value. For example, the system can issue an alert if the number of identified drinking events decreases by 5, 10, 15, 20, 25, 30, 35, 40, 45, 50, 60, 70, or 80 percent or more, or a value falling within a range between any of the foregoing.

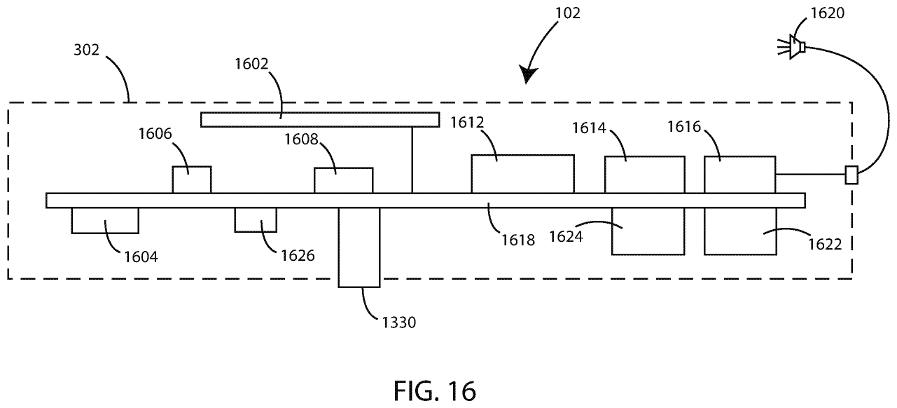

Referring now to FIG. 16, a schematic block diagram of components of an ear-wearable device is shown in accordance with various embodiments herein. The block diagram of FIG. 16 represents a generic ear-wearable device for purposes of illustration. The ear-wearable device 102 shown in FIG. 16 includes several components electrically connected to a flexible mother circuit 1618 (e.g., flexible mother board) which is disposed within housing 302. A power supply 1604 or power supply circuit can include a battery and circuitry to regulate power and can be electrically connected to the flexible mother circuit 1618 and provides power to the various components of the ear-wearable device 102. One or more microphones 1606 are electrically connected to the flexible mother circuit 1618, which provides electrical communication between the microphones 1606 and a digital signal processor (DSP) 1612. Among other components, the DSP 1612 incorporates or is coupled to audio signal processing circuitry configured to implement various functions described herein. A sensor package 1614 can be coupled to the DSP 1612 via the flexible mother circuit 1618. The sensor package 1614 can include one or more different specific types of sensors such as those described in greater detail below. One or more user input devices 1330 (e.g., on/off, volume, mic directional settings) can be electrically coupled to the DSP 1612 and/or other components via the flexible mother circuit 1618.

An audio output device 1616 is electrically connected to the DSP 1612 via the flexible mother circuit 1618. In some embodiments, the audio output device 1616 comprises an electroacoustic transducer or speaker (coupled to an amplifier). In other embodiments, the audio output device 1616 comprises an amplifier coupled to an external receiver 1620 adapted for positioning within an ear of a wearer. The external receiver 1620 can include an electroacoustic transducer, speaker, or loudspeaker.

The ear-wearable device 102 may incorporate a communication device 1608 coupled to the flexible mother circuit 1618 and to an antenna 1602 directly or indirectly via the flexible mother circuit 1618. The communication device 1608 can be a BLUETOOTH® transceiver, such as a BLE (BLUETOOTH® low energy) transceiver or other transceiver(s) (e.g., an IEEE 802.11 compliant device). The communication device 1608 can be configured to communicate with one or more external devices, such as those discussed previously, in accordance with various embodiments. In various embodiments, the communication device 1608 can be configured to communicate with an external visual display device such as a smart phone, a video display screen, a tablet, a computer, or the like.

In some embodiments, ear-wearable devices 102 of the present disclosure can incorporate an antenna arrangement coupled to a high-frequency radio, such as a 2.4 GHz radio. The radio can conform to an IEEE 802.11 (e.g., WIFI®) or BLUETOOTH® (e.g., BLE, BLUETOOTH® 4.2 or 5.0) specification, for example. It is understood that ear-wearable devices of the present disclosure can employ other radios, such as a 900 MHz radio or radios operating at other frequencies or frequency bands. Ear-wearable device of the present disclosure can also include hardware, such as one or more antennas, for NFMI or NFC wireless communications. Ear-wearable devices of the present disclosure can be configured to receive streaming audio (e.g., digital audio data or files) from an electronic or digital source.

Ear-wearable devices 102 of the present disclosure can be configured to receive streaming audio (e.g., digital audio data or files) from an electronic or digital source. Representative electronic/digital sources (also referred to herein as accessory devices) include an assistive listening system, a TV streamer, a radio, a smartphone, a cell phone/entertainment device (CPED) or other electronic device that serves as a source of digital audio data or files. Systems herein can also include these types of accessory devices as well as other types of devices.

In various embodiments, the ear-wearable device 102 can also include a control circuit 1622 and a memory storage device 1624. The control circuit 1622 can be in electrical communication with other components of the device. In some embodiments, a clock circuit 1626 can be in electrical communication with the control circuit. The control circuit 1622 can execute various operations, such as those described herein. The control circuit 1622 can include various components including, but not limited to, a microprocessor, a microcontroller, an FPGA (field-programmable gate array) processing device, an ASIC (application specific integrated circuit), or the like. The memory storage device 1624 can include both volatile and non-volatile memory. The memory storage device 1624 can include ROM, RAM, flash memory, EEPROM, SSD devices, NAND chips, and the like. The memory storage device 1624 can be used to store data from sensors as described herein and/or processed data generated using data from sensors as described herein.

It will be appreciated that various of the components described in FIG. 16 can be associated with separate devices and/or accessory devices to the ear-wearable device. By way of example, microphones can be associated with separate devices and/or accessory devices. Similarly, audio output devices can be associated with separate devices and/or accessory devices to the ear-wearable device.

Accessory devices herein can include various different components. In some embodiments, the accessory device can be a personal communications device, such as a smart phone. However, the accessory device can also be other things such as a secondary wearable device, a handheld computing device, a dedicated location determining device (such as a handheld GPS unit), or the like.

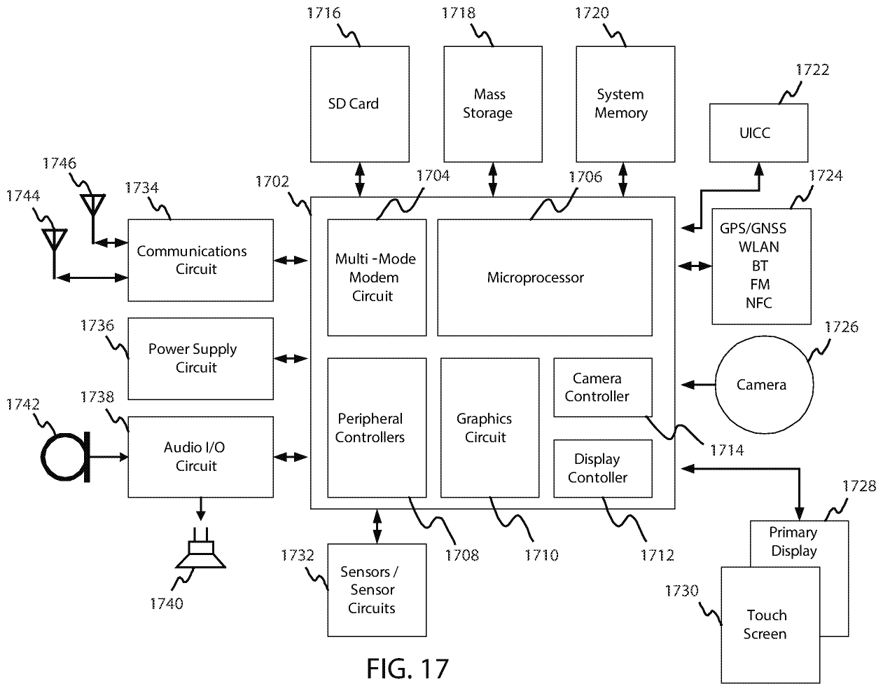

Referring now to FIG. 17, a schematic block diagram is shown of components of an accessory device 104 (which could be a personal communications device or another type of accessory device) in accordance with various embodiments herein. This block diagram is just provided by way of illustration and it will be appreciated that accessory devices can include greater or lesser numbers of components. The accessory device in this example can include a control circuit 1702. The control circuit 1702 can include various components which may or may not be integrated. In various embodiments, the control circuit 1702 can include a microprocessor 1706, which could also be a microcontroller, FPGA, ASIC, or the like. The control circuit 1702 can also include a multi-mode modem circuit 1704 which can provide communications capability via various wired and wireless standards. The control circuit 1702 can include various peripheral controllers 1708. The control circuit 1702 can also include various sensors/sensor circuits 1732. The control circuit 1702 can also include a graphics circuit 1710, a camera controller 1714, and a display controller 1712. In various embodiments, the control circuit 1702 can interface with an SD card 1716, mass storage 1718, and system memory 1720. In various embodiments, the control circuit 1702 can interface with universal integrated circuit card (UICC) 1722. A spatial location determining circuit can be included and can take the form of an integrated circuit 1724 that can include components for receiving signals from GPS, GLONASS, BeiDou, Galileo, SBAS, WLAN, BT, FM, NFC type protocols, 5G picocells, or E911. In various embodiments, the accessory device can include a camera 1726. In various embodiments, the control circuit 1702 can interface with a primary display 1728 that can also include a touch screen 1730. In various embodiments, an audio I/O circuit 1738 can interface with the control circuit 1702 as well as a microphone 1742 and a speaker 1740. In various embodiments, a power supply or power supply circuit 1736 can interface with the control circuit 1702 and/or various other circuits herein in order to provide power to the system. In various embodiments, a communications circuit 1734 can be in communication with the control circuit 1702 as well as one or more antennas (1744, 1746).

It will be appreciated that in various embodiments herein, a device or a system can be used to detect a pattern or patterns (such as patterns of data from sensors) indicative of a state of dehydration. Also, it will be appreciated that in various embodiments herein, a device or a system can be used to detect a pattern or patterns indicative of a specific event, such as drinking which can impact an individual's present state of hydration. Such patterns can be detected in various ways. Some techniques are described elsewhere herein, but some further examples will now be described.

As merely one example, one or more sensors can be operatively connected to a controller (such as the control circuit described in FIG. 17) or another processing resource (such as a processor of another device or a processing resource in the cloud). The controller or other processing resource can be adapted to receive data representative of a characteristic of the subject from one or more of the sensors and/or determine statistics of the subject over a monitoring time period based upon the data received from the sensor. As used herein, the term “data” can include a single datum or a plurality of data values or statistics. The term “statistics” can include any appropriate mathematical calculation or metric relative to data interpretation, e.g., probability, confidence interval, distribution, range, or the like. Further, as used herein, the term “monitoring time period” means a period of time over which characteristics of the subject are measured and statistics are determined. The monitoring time period can be any suitable length of time, e.g., 10 seconds, 30 seconds, 1 minute, 10 minutes, 30 minutes, 1 hour, 1 day, 1 week, etc., or a range of time between any of the foregoing time periods.

Any suitable technique or techniques can be utilized to determine statistics for the various data from the sensors, e.g., direct statistical analyses of time series data from the sensors, differential statistics, comparisons to baseline or statistical models of similar data, etc. Such techniques can be general or individual-specific and represent long-term or short-term behavior. These techniques could include standard pattern classification methods such as Gaussian mixture models, clustering as well as Bayesian approaches, neural network models and deep learning.

Further, in some embodiments, the controller can be adapted to compare data, data features, and/or statistics against various other patterns, which could be prerecorded patterns (baseline patterns) of the particular individual wearing an ear-wearable device herein, prerecorded patterns (group baseline patterns) of a group of individuals wearing ear-wearable devices herein, one or more predetermined patterns that serve as positive example patterns (such as patterns indicative of hydration/dehydration states), negative example patterns, or the like. As merely one scenario, if a pattern is detected in an individual that exhibits similarity crossing a threshold value to a positive example pattern or substantial similarity to that pattern, then that can be taken as an indication of the presence of a level of hydration/dehydration associated with the positive example pattern. Positive and/or negative example patterns can be stored or accessed for use covering those items to be detected in accordance with embodiments herein including, but not limited to, states of dehydration/hydration, clinical signs of dehydration, events impacting dehydration such as drinking and activity, relevant events with characteristic sounds such as the licking or smacking of lips, environmental conditions impacting dehydration such as weather, temperature, humidity, and the like and other items discussed elsewhere herein.

Similarity and dissimilarity can be measured directly via standard statistical metrics such normalized Z-score, or similar multidimensional distance measures (e.g. Mahalanobis or Bhattacharyya distance metrics), or through similarities of modeled data and machine learning. These techniques can include standard pattern classification methods such as Gaussian mixture models, clustering as well as Bayesian approaches, neural network models, and deep learning.

As used herein the term “substantially similar” means that, upon comparison, the sensor data are congruent or have statistics fitting the same statistical model, each with an acceptable degree of confidence. The threshold for the acceptability of a confidence statistic may vary depending upon the subject, sensor, sensor arrangement, type of data, context, condition, etc.

The statistics associated with the hydration/dehydration status of an individual over the monitoring time period, can be determined by utilizing any suitable technique or techniques, e.g., standard pattern classification methods such as Gaussian mixture models, clustering, hidden Markov models, as well as Bayesian approaches, neural network models, and deep learning.

### Methods

Many different methods are contemplated herein, including, but not limited to, methods of making devices and systems herein, methods of using devices and systems herein, methods of monitoring an individual for hydration levels or dehydration, methods of monitoring ear canal humidity, and the like. Aspects of system/device operation described elsewhere herein can be performed as operations of one or more methods in accordance with various embodiments herein.

Specifically, in various embodiments herein, a method of monitoring an individual for hydration levels or dehydration using an ear-wearable monitoring system is included. The method can include gathering signals with a microphone, gathering signals with a sensor package, and processing signals of the microphone and the sensor package to detect clinical symptoms of hydration levels or dehydration.

In an embodiment of the method, the clinical symptoms of hydration levels or dehydration including one or more of rapid shallow breathing, increased pulse, low blood pressure, dizziness, change in voice quality, increased temperature.

In an embodiment, the method can further include processing signals specifically of the microphone to detect signs of hydration levels or dehydration. In an embodiment of the method, the signs of hydration levels or dehydration include dysphonia. In an embodiment of the method, the changes in voice quality include changes in tonal properties. In an embodiment of the method, the signs of hydration levels or dehydration include changes in voice pitch and/or tremor. In an embodiment of the method, the signs of hydration levels or dehydration include smacking or licking lips.

In an embodiment, the method can further include issuing an alert when hydration levels or dehydration clinical symptoms cross a threshold value. In some embodiments, the threshold values are predetermined. However, in some embodiments of the method, the threshold value is dynamically set based on factors including one or more of an ambient temperature, an ambient humidity, and activity levels of the device wearer.

In various embodiments, the method can further include identifying drinking events based at least in part on signals from the microphone and record the same. In an embodiment of the method, identifying drinking events based at least in part on signals from the microphone and record the same further comprises issuing an alert if a number of identified drinking events over a defined time period change by at least a threshold value.

In an embodiment, the method can further include issuing an alert when dehydration clinical symptoms cross a threshold value for at least a threshold period of time.

In an embodiment, the method can further include classifying an observed pattern representing signals from the microphone and the sensor package into a scale of dehydration severities using a machine learning derived algorithm.

In an embodiment, a method of monitoring ear canal humidity to detect dehydration of an individual is included. The method can include sealing off a portion of an individual's ear canal, measuring humidity within the sealed off portion of the ear canal, and evaluating the measured humidity to detect dehydration.

In an embodiment, the method can further include issuing an alert when humidity values cross a threshold value. In an embodiment, the method can further include issuing an alert when humidity values cross a threshold value for at least a threshold period of time. In some embodiments the threshold value can be predetermined. In other embodiments of the method, the threshold value is dynamically set based on factors including one or more of an ambient temperature, an ambient humidity, and activity levels of the device wearer.

### Sensors

Ear-wearable devices herein can include one or more sensor packages (including one or more discrete or integrated sensors) to provide data. The sensor package can comprise one or a multiplicity of sensors. In some embodiments, the sensor packages can include one or more motion sensors (or movement sensors) amongst other types of sensors. Motion sensors herein can include inertial measurement units (IMU), accelerometers, gyroscopes, barometers, altimeters, and the like. The IMU can be of a type disclosed in commonly owned U.S. Pat. No. 9,848,273, which is incorporated herein by reference. In some embodiments, electromagnetic communication radios or electromagnetic field sensors (e.g., telecoil, NFMI, TMR, GMR, etc.) sensors may be used to detect motion or changes in position. In various embodiments, the sensor package can include a magnetometer. In some embodiments, biometric sensors may be used to detect body motions or physical activity. Motions sensors can be used to track movement of a patient in accordance with various embodiments herein.

In some embodiments, the motion sensors can be disposed in a fixed position with respect to the head of a patient, such as worn on or near the head or ears. In some embodiments, the operatively connected motion sensors can be worn on or near another part of the body such as on a wrist, arm, or leg of the patient.

According to various embodiments, the sensor package can include one or more of an IMU, and accelerometer (3, 6, or 9 axis), a gyroscope, a barometer, an altimeter, a magnetometer, a magnetic sensor, an eye movement sensor, a pressure sensor, an acoustic sensor, a telecoil, a heart rate sensor, a global positioning system (GPS), a microphone, an acoustic sensor, a wireless radio antenna, an air quality sensor, an optical sensor, a light sensor, an image sensor, a temperature sensor, a physiological sensor such as a blood pressure sensor, an oxygen saturation sensor, a blood glucose sensor (optical or otherwise), a galvanic skin response sensor, a cortisol level sensor (optical or otherwise), an electrocardiogram (ECG) sensor, electroencephalography (EEG) sensor which can be a neurological sensor, eye movement sensor (e.g., electrooculogram (EOG) sensor), myographic potential electrode sensor (EMG), a heart rate monitor, a pulse oximeter or oxygen saturation sensor (SpO2), blood perfusion sensor, hydrometer, sweat sensor, humidity sensor, cerumen sensor, pupillometry sensor, hematocrit sensor, or the like.

In some embodiments, the sensor package can be part of an ear-wearable device. However, in some embodiments, the sensor packages can include one or more additional sensors that are external to an ear-wearable device. For example, various of the sensors described above can be part of a wrist-worn or ankle-worn sensor package, or a sensor package supported by a chest strap. In some embodiments, sensors herein can be disposable sensors that are adhered to the device wearer (“adhesive sensors”) and that provide data to the ear-wearable device or another component of the system.

Data produced by the sensor(s) of the sensor package can be operated on by a processor of the device or system.

As used herein the term “inertial measurement unit” or “IMU” shall refer to an electronic device that can generate signals related to a body's specific force and/or angular rate. IMUs herein can include one or more accelerometers (3, 6, or 9 axis) to detect linear acceleration and a gyroscope to detect rotational acceleration and/or velocity. In some embodiments, an IMU can also include a magnetometer to detect a magnetic field.

An eye movement sensor herein be, for example, an electrooculographic (EOG) sensor, such as an EOG sensor disclosed in commonly owned U.S. Pat. No. 9,167,356, which is incorporated herein by reference. The pressure sensor can be, for example, a MEMS-based pressure sensor, a piezo-resistive pressure sensor, a flexion sensor, a strain sensor, a diaphragm-type sensor and the like.

A temperature sensor herein can be, for example, a thermistor (thermally sensitive resistor), a resistance temperature detector, a thermocouple, a semiconductor-based sensor, an infrared sensor, or the like.

A blood pressure sensor herein can be, for example, a pressure sensor. The heart rate sensor can be, for example, an electrical signal sensor, an acoustic sensor, a pressure sensor, an infrared sensor, an optical sensor, or the like.

A oxygen saturation sensor (such as a blood oximetry sensor) herein can be, for example, an optical sensor, an infrared sensor, a visible light sensor, or the like.

An electrical signal sensor herein can include two or more electrodes and can include circuitry to sense and record electrical signals including sensed electrical potentials and the magnitude thereof (according to Ohm's law where V=IR) as well as measure impedance from an applied electrical potential.

A humidity sensor herein can be, for example, a capacitive humidity sensor, a resistive humidity sensor, a thermal conductivity humidity sensor, or the like.

It will be appreciated that the sensor package can include one or more sensors that are external to the ear-wearable device. In addition to the external sensors discussed hereinabove, the sensor package can comprise a network of body sensors (such as those listed above) that sense movement of a multiplicity of body parts (e.g., arms, legs, torso).

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An ear-wearable hydration level monitoring system comprising:
a control circuit;
a microphone, wherein the microphone is in electrical communication with the control circuit;
a power supply, wherein the power supply is in electrical communication with the control circuit; and
a sensor package, wherein the sensor package is in electrical communication with the control circuit;
wherein the ear-wearable hydration level monitoring system is configured to process signals of one or more sensors of the sensor package to detect clinical symptoms of hydration levels.

2. The ear-wearable hydration level monitoring system of any of claims 1 and 3-20, the sensor package comprising a motion sensor.

3. The ear-wearable hydration level monitoring system of any of claims 1-2 and 4-20, the sensor package comprising at least one selected from the group consisting of a photoplethysmography sensor, a temperature sensor, and a motion sensor.

4. The ear-wearable hydration level monitoring system of any of claims 1-3 and 5-20, the sensor package comprising at least one selected from the group consisting of a photoplethysmography (PPG) sensor, a electrocardiography (ECG) sensor, a temperature sensor, an electromyography (EMG) sensor, a motion sensor, an electroencephalography (EEG) sensor, and a glucose sensor.

5. The ear-wearable hydration level monitoring system of any of claims 1-4 and 6-20, wherein the clinical symptoms of hydration levels including one or more of rapid shallow breathing, increased pulse, low blood pressure, dizziness, change in voice quality, increased temperature.

6. The ear-wearable hydration level monitoring system of any of claims 1-5 and 7-20, wherein the changes in voice quality include changes in tonal properties.

7. The ear-wearable hydration level monitoring system of any of claims 1-6 and 8-20, wherein the ear-wearable hydration level monitoring system is configured to receive data from at least one external sensor.

8. The ear-wearable hydration level monitoring system of any of claims 1-7 and 9-20, wherein the external sensor is selected from the group consisting of a humidity sensor, an ambient temperature sensor, a weight sensor, and a sensor disposed on a charging device for the ear-wearable hydration level monitoring system.

9. The ear-wearable hydration level monitoring system of any of claims 1-8 and 10-20, wherein the ear-wearable hydration level monitoring system is configured to process signals of the microphone to detect signs of hydration levels.

10. The ear-wearable hydration level monitoring system of any of claims 1-9 and 11-20, wherein the signs of hydration levels include smacking or licking lips.

11. The ear-wearable hydration level monitoring system of any of claims 1-10 and 12-20, wherein the signs of hydration levels include pitch tremor.

12. The ear-wearable hydration level monitoring system of any of claims 1-11 and 13-20, wherein the signs of hydration levels include rapid shallow breathing.

13. The ear-wearable hydration level monitoring system of any of claims 1-12 and 14-20, wherein the signs of hydration levels include dysphonia.

14. The ear-wearable hydration level monitoring system of any of claims 1-13 and 15-20, wherein the ear-wearable hydration level monitoring system is configured to issue an alert when hydration level clinical symptoms cross a threshold value.

15. The ear-wearable hydration level monitoring system of any of claims 1-14 and 16-20, wherein the threshold value is dynamically set based on factors including one or more of an ambient temperature, an ambient humidity, and activity levels of the device wearer.

16. The ear-wearable hydration level monitoring system of any of claims 1-15 and 17-20, wherein the ear-wearable hydration level monitoring system is configured to identify drinking events based at least in part on signals from the microphone and record the same.

17. The ear-wearable hydration level monitoring system of any of claims 1-16 and 18-20, wherein the ear-wearable hydration level monitoring system is configured to issue an alert if a number of identified drinking events over a defined time period change by at least a threshold value.

18. The ear-wearable hydration level monitoring system of any of claims 1-17 and 19-20, wherein the ear-wearable hydration level monitoring system is configured to issue an alert when hydration level clinical symptoms cross a threshold value for at least a threshold period of time.

19. The ear-wearable hydration level monitoring system of any of claims 1-18 and 20, further comprising:
a first unit, wherein the first unit is configured to be wearable about a first ear; and
a second unit, wherein the second unit is configured to be wearable about a second ear;
wherein signals are exchanged between the first unit and the second unit.

20. The ear-wearable hydration level monitoring system of any of claims 1-19, wherein the ear-wearable hydration level monitoring system is configured to classify an observed pattern representing signals from the microphone and the sensor package into a scale of hydration levels using a machine learning derived algorithm.

21. An ear-wearable hydration level monitoring system comprising:
a control circuit;
a microphone, wherein the microphone is in electrical communication with the control circuit;
a power supply, wherein the power supply is in electrical communication with the control circuit;
a sealing member, wherein the sealing member is attached to a structure; and
a humidity sensor, wherein the humidity sensor is attached to the structure; and
wherein the humidity sensor is configured to measure humidity within an ear canal of a wearer of the ear-wearable hydration level monitoring system between the sealing dome and a tympanic membrane of the wearer.

22. The ear-wearable hydration level monitoring system of any of claims 21 and 23-34, wherein the ear-wearable hydration level monitoring system is configured to receive data from at least one external sensor.

23. The ear-wearable hydration level monitoring system of any of claims 21-22 and 24-34, wherein the external sensor is selected from the group consisting of a humidity sensor, an ambient temperature sensor, a weight sensor, and a sensor disposed on a charging device for the ear-wearable hydration level monitoring system.

24. The ear-wearable hydration level monitoring system of any of claims 21-23 and 25-34, the sealing member comprising a sealing dome.

25. The ear-wearable hydration level monitoring system of any of claims 21-24 and 26-34, the sealing member comprising a sealing baffle.

26. The ear-wearable hydration level monitoring system of any of claims 21-25 and 27-34,
wherein the sealing baffle is mounted on a receiver;
an ear-wearable hydration level monitoring system further comprising the receiver.

27. The ear-wearable hydration level monitoring system of any of claims 21-26 and 28-34, further comprising a sensor package, wherein the sensor package is in electrical communication with the control circuit.

28. The ear-wearable hydration level monitoring system of any of claims 21-27 and 29-34, the sensor package comprising a motion sensor.

29. The ear-wearable hydration level monitoring system of any of claims 21-28 and 30-34, the sensor package comprising at least one selected from the group consisting of a photoplethysmography sensor, a temperature sensor, and a motion sensor.

30. The ear-wearable hydration level monitoring system of any of claims 21-29 and 31-34, the sensor package comprising at least one selected from the group consisting of a photoplethysmography (PPG) sensor, a electrocardiography (ECG) sensor, a temperature sensor, an electromyography (EMG) sensor, a motion sensor, an electroencephalography (EEG) sensor, and a glucose sensor.

31. The ear-wearable hydration level monitoring system of any of claims 21-30 and 32-34, wherein the ear-wearable hydration level monitoring system is configured to issue an alert when ear canal humidity crosses a threshold value.

32. The ear-wearable hydration level monitoring system of any of claims 21-31 and 33-34, wherein the threshold value is dynamically set based on factors including one or more of an ambient temperature, an ambient humidity, and activity levels of the device wearer.

33. The ear-wearable hydration level monitoring system of any of claims 21-32 and 34, wherein the ear-wearable hydration level monitoring system is configured to identify drinking events based at least in part on signals from the microphone and record the same.

34. The ear-wearable hydration level monitoring system of any of claims 21-33, wherein the ear-wearable hydration level monitoring system is configured to issue an alert if a number of identified drinking events over a defined time period change by at least a threshold value.

35. A method of monitoring an individual for hydration levels using an ear-wearable monitoring system comprising:
gathering signals with a microphone;
gathering signals with a sensor package; and
processing signals of the microphone and the sensor package to detect clinical symptoms of hydration levels.

36. The method of any of claims 35 and 37-48, wherein the clinical symptoms of hydration levels including one or more of rapid shallow breathing, increased pulse, low blood pressure, dizziness, change in voice quality, increased temperature.

37. The method of any of claims 35-36 and 38-48, wherein the changes in voice quality include changes in tonal properties.

38. The method of any of claims 35-37 and 39-48, further comprising processing signals of the microphone to detect signs of hydration levels.

39. The method of any of claims 35-38 and 40-48, wherein the signs of hydration levels include dysphonia.

40. The method of any of claims 35-39 and 41-48, wherein the signs of hydration levels include rapid shallow breathing.

41. The method of any of claims 35-40 and 42-48, wherein the signs of hydration levels include pitch tremor.

42. The method of any of claims 35-41 and 43-48, wherein the signs of hydration levels include smacking or licking lips.

43. The method of any of claims 35-42 and 44-48, further comprising issuing an alert when hydration levels clinical symptoms cross a threshold value.

44. The method of any of claims 35-43 and 45-48, wherein the threshold value is dynamically set based on factors including one or more of an ambient temperature, an ambient humidity, and activity levels of the device wearer.

45. The method of any of claims 35-44 and 46-48, further comprising identifying drinking events based at least in part on signals from the microphone and record the same.

46. The method of any of claims 35-45 and 47-48, wherein identifying drinking events based at least in part on signals from the microphone and record the same further comprises issuing an alert if a number of identified drinking events over a defined time period change by at least a threshold value.

47. The method of any of claims 35-46 and 48, further comprising issuing an alert when hydration levels clinical symptoms cross a threshold value for at least a threshold period of time.

48. The method of any of claims 35-47, further comprising classifying an observed pattern representing signals from the microphone and the sensor package into a scale of hydration levels severities using a machine learning derived algorithm.

49. A method of monitoring ear canal humidity to detect hydration levels of an individual comprising:
sealing off a portion of an individual's ear canal;
measuring humidity within the sealed off portion of the ear canal; and
evaluating the measured humidity to detect hydration levels.

50. The method of any of claims 49 and 51-64, further comprising issuing an alert when humidity values cross a threshold value.

51. The method of any of claims 49-50 and 52-64, wherein the threshold value is dynamically set based on factors including one or more of an ambient temperature, an ambient humidity, and activity levels of the device wearer.

52. The method of any of claims 49-51 and 53-64, further comprising issuing an alert when humidity values cross a threshold value for at least a threshold period of time.

53. The method of any of claims 49-52 and 54-64, further comprising identifying drinking events based at least in part on signals from a microphone and record the same.

54. The method of any of claims 49-53 and 55-64, further comprising issuing an alert if a number of identified drinking events over a defined time period change by at least a threshold value.

55. The method of any of claims 49-54 and 56-64, further comprising gathering signals with a microphone.

56. The method of any of claims 49-55 and 57-64, further comprising gathering signals with a sensor package.

57. The method of any of claims 49-56 and 58-64, further comprising processing signals of the microphone and the sensor package to detect clinical symptoms of hydration levels.

58. The method of any of claims 49-57 and 59-64, wherein the clinical symptoms of hydration levels including one or more of rapid shallow breathing, increased pulse, low blood pressure, dizziness, change in voice quality, increased temperature.

59. The method of any of claims 49-58 and 60-64, wherein the changes in voice quality include changes in tonal properties.

60. The method of any of claims 49-59 and 61-64, further comprising processing signals of the microphone to detect signs of hydration levels.

61. The method of any of claims 49-60 and 62-64, wherein the signs of hydration levels include smacking or licking lips.

62. The method of any of claims 49-61 and 63-64, wherein the signs of hydration levels include pitch tremor.

63. The method of any of claims 49-62 and 64, wherein the signs of hydration levels include rapid shallow breathing.

64. The method of any of claims 49-63, wherein the signs of hydration levels include dysphonia.

65. An ear-wearable dehydration monitoring system comprising:
a control circuit;
a microphone, wherein the microphone is in electrical communication with the control circuit;
a power supply, wherein the power supply is in electrical communication with the control circuit; and
a sensor package, wherein the sensor package is in electrical communication with the control circuit;
wherein the ear-wearable dehydration monitoring system is configured to process signals of one or more sensors of the sensor package to detect clinical symptoms of dehydration.

66. The ear-wearable dehydration monitoring system of any of claims 65 and 67-84, the sensor package comprising a motion sensor.

67. The ear-wearable dehydration monitoring system of any of claims 65-66 and 68-84, the sensor package comprising at least one selected from the group consisting of a photoplethysmography sensor, a temperature sensor, and a motion sensor.

68. The ear-wearable dehydration monitoring system of any of claims 65-67 and 69-84, the sensor package comprising at least one selected from the group consisting of a photoplethysmography (PPG) sensor, a electrocardiography (ECG) sensor, a temperature sensor, an electromyography (EMG) sensor, a motion sensor, an electroencephalography (EEG) sensor, and a glucose sensor.

69. The ear-wearable dehydration monitoring system of any of claims 65-68 and 70-84, wherein the clinical symptoms of dehydration including one or more of rapid shallow breathing, increased pulse, low blood pressure, dizziness, change in voice quality, increased temperature.

70. The ear-wearable dehydration monitoring system of any of claims 65-69 and 71-84, wherein the changes in voice quality include changes in tonal properties.

71. The ear-wearable dehydration monitoring system of any of claims 65-70 and 72-84, wherein the ear-wearable dehydration monitoring system is configured to receive data from at least one external sensor.

72. The ear-wearable dehydration monitoring system of any of claims 65-71 and 73-84, wherein the external sensor is selected from the group consisting of a humidity sensor, an ambient temperature sensor, a weight sensor, and a sensor disposed on a charging device for the ear-wearable dehydration monitoring system.

73. The ear-wearable dehydration monitoring system of any of claims 65-72 and 74-84, wherein the ear-wearable dehydration monitoring system is configured to process signals of the microphone to detect signs of dehydration.

74. The ear-wearable dehydration monitoring system of any of claims 65-73 and 75-84, wherein the signs of dehydration include smacking or licking lips.

75. The ear-wearable dehydration monitoring system of any of claims 65-74 and 76-84, wherein the signs of dehydration include pitch tremor.

76. The ear-wearable dehydration monitoring system of any of claims 65-75 and 77-84, wherein the signs of dehydration include rapid shallow breathing.

77. The ear-wearable dehydration monitoring system of any of claims 65-76 and 78-84, wherein the signs of dehydration include dysphonia.

78. The ear-wearable dehydration monitoring system of any of claims 65-77 and 79-84, wherein the ear-wearable dehydration monitoring system is configured to issue an alert when dehydration clinical symptoms cross a threshold value.

79. The ear-wearable dehydration monitoring system of any of claims 65-78 and 80-84, wherein the threshold value is dynamically set based on factors including one or more of an ambient temperature, an ambient humidity, and activity levels of the device wearer.

80. The ear-wearable dehydration monitoring system of any of claims 65-79 and 81-84, wherein the ear-wearable dehydration monitoring system is configured to identify drinking events based at least in part on signals from the microphone and record the same.

81. The ear-wearable dehydration monitoring system of any of claims 65-80 and 82-84, wherein the ear-wearable dehydration monitoring system is configured to issue an alert if a number of identified drinking events over a defined time period change by at least a threshold value.

82. The ear-wearable dehydration monitoring system of any of claims 65-81 and 83-84, wherein the ear-wearable dehydration monitoring system is configured to issue an alert when dehydration clinical symptoms cross a threshold value for at least a threshold period of time.

83. The ear-wearable dehydration monitoring system of any of claims 65-82 and 84, further comprising:
a first unit, wherein the first unit is configured to be wearable about a first ear; and
a second unit, wherein the second unit is configured to be wearable about a second ear;
wherein signals are exchanged between the first unit and the second unit.

84. The ear-wearable dehydration monitoring system of any of claims 65-83, wherein the ear-wearable dehydration monitoring system is configured to classify an observed pattern representing signals from the microphone and the sensor package into a scale of dehydration severities using a machine learning derived algorithm.
